SEQUENCE LISTING (7 sequences)

SEQ ID NO: 1 - protein, 75 aa, Homo sapiens
MTEYKLVVVGAGGVGKSALTIQLIQNHFVDEYDPTIEGVDDAFYTLVREIRKHKEKMSKDGKKKKKKSKTKCVIM

SEQ ID NO: 2 - protein, 21 aa, synthetic construct
YKLVVVGACGVGKSALTIQLI

SEQ ID NO: 3 - protein, 21 aa, synthetic construct
YKLVVVGAVGVGKSALTIQLI

SEQ ID NO: 4 - protein, 21 aa, synthetic construct
YKLVVVGADGVGKSALTIQLI

SEQ ID NO: 5 - protein, 21 aa, synthetic construct
YKLVVVGAAGVGKSALTIQLI

SEQ ID NO: 6 - protein, 21 aa, synthetic construct
YKLVVVGAGDVGKSALTIQLI

SEQ ID NO: 7 - protein, 21 aa, synthetic construct
YKLVVVGARGVGKSALTIQLI

(19) United States
(12) Patent Application Publication
(10) Pub. No.: US 2025/0332237 A1
(43) Pub. Date: Oct. 30, 2025

(54) NEOANTIGEN VACCINES FOR CANCER PREVENTION

(71) Applicant: The Johns Hopkins University, Baltimore, MD (US)

(72) Inventors: Elizabeth A. Jaffee, Baltimore, MD (US); Neeha Zaidi, Baltimore, MD (US); Mark Yarchoan, Baltimore, MD (US); Amanda Huff, Baltimore, MD (US)

(21) Appl. No.: 18/944,694

(22) Filed: Nov. 12, 2024

Related U.S. Application Data
(63) Continuation of application No. PCT/US23/22136, filed on May 12, 2023.
(60) Provisional application No. 63/341,206, filed on May 12, 2022.

(51) Int. Cl. A61K 39/00; A61K 38/17; A61K 39/395; A61P 37/04

(52) U.S. Cl. CPC A61K 39/001154; A61K 38/1774; A61K 39/3955; A61P 37/04; A61K 2039/55561

(57) ABSTRACT

Vaccines targeting neoantigens or tumor-specific proteins for the prevention of cancer include peptides having one or more mutations that correspond to oncogene sequences.

### CROSS-REFERENCES TO RELATED APPLICATION

This application is a continuation of PCT Application No. PCT/US23/22136, filed May 12, 2023, which claims the benefit of priority of U.S. Provisional Application No. 63/341,206 filed on May 12, 2022, which applications are is incorporated herein by reference in its entirety and for all purposes.

### STATEMENT REGARDING FEDERALLY SPONSORED RESEARCH

This disclosure was made with government support under grant numbers CA248624, CA062924 and CA247886 awarded by the National Institutes of Health. The government has certain rights in this disclosure.

### SEQUENCE LISTING

The instant application contains a Sequence Listing which has been submitted electronically in XML file format and is hereby incorporated by reference in its entirety. Said XML copy, created on Jul. 26, 2023, is named 348358_13902_SL.xml and is 7,222 bytes in size.”

### BACKGROUND

Neoantigens are emerging as a very strong option to advance personalized cancer medicine, as they have tremendous potential to effect cancer treatments that provide truly individualized immunotherapies.

Neoantigens are the result of mutations in the somatic DNA of tumors and, as such, represent a form of personalized therapy. In contrast to shared tumor antigens which are selectively expressed or over-expressed in tumors in many individuals (but still may be expressed in normal cells), neoantigens contain tumor-specific and/or patient-specific mutations and have the potential to uniquely mark a tumor for destruction while avoiding self-tolerance.

As a result of these and other mutations or modifications, neoantigens contain predicted epitopes (B cell and T cell) that are unique to each patient. Neoantigens, and the neoepitopes contained therein, may or may not be immunogenic when injected as a vaccine, therefore selecting the appropriate formulation for immunization is crucial for ensuring optimal immunogenicity. Additionally, since each peptide pool is unique to each patient, the process of identifying and then formulating the neoantigens and/or neoepitopes into an appropriate vaccine formulation within a reasonable time frame is a significant consideration in respect of their ultimate use in patient therapy. Each peptide pool will contain different peptides with different properties which may require optimization, particularly if the vaccine formulation is not sufficient to handle weakly immunogenic antigens.

### SUMMARY

We now provide immunogenic compositions and vaccines comprising one or more long peptides corresponding to an oncogene, for example mutated KRAS that is expressed in the majority of pancreas cancers and is present in the earliest stages of pancreatic cancer development. These vaccines target the most common neoantigens that are critical to the growth of cancer cells.

The therapeutic efficacy of these vaccines has been demonstrated in human subjects, including human subjects suffering from pancreatic cancer. See the examples which follows.

Accordingly, in certain aspects, a vaccine comprises one or more peptides wherein at least one of the one or more peptides comprises a peptide of a RAS oncogene, wherein the peptide comprises one or more mutations. In certain embodiments, at least one of the one or more peptides comprise at least 10 amino acid residues. In certain embodiments, at least one of the one or more peptides comprise at least 15 amino acid residues. In certain embodiments, at least one of the one or more peptides comprise at least 20 amino acid residues.

In certain embodiments, the RAS oncogene peptide is a mutated KRAS peptide.

In certain embodiments, the mutated KRAS peptide comprises one or more mutations at position 9, 10 or the combination thereof. In certain embodiments, the vaccine further comprises the mutated KRAS peptide which comprises one or more mutations comprising G12C, G12V, G12D, G12A, G12R, G13D or combinations thereof.

In certain embodiments, the mutated KRAS peptides comprise one or more of SEQ ID NOs: 1-7 or combinations thereof.

In certain embodiments, the vaccine further comprises an immunostimulant. In certain embodiments, the immunostimulant comprises polyinosinic: polycytidylic acid (poly(I:C), poly-ICLC, derivatives thereof, or combinations thereof. In certain embodiments, the vaccine further comprises a pharmaceutical composition. In certain embodiments, the vaccine further comprises an adjuvant.

In certain aspects, a peptide vaccine comprises a plurality of KRAS peptides comprising one or more mutations. In certain embodiments, the KRAS peptides comprise at least 10 amino acid residues. In certain embodiments, the KRAS peptides comprise at least 15 amino acid residues. In certain embodiments, the KRAS peptides comprise at least 20 amino acid residues. In certain embodiments, the mutated KRAS peptide comprises one or more mutations comprising G12C, G12V, G12D, G12A, G12R, G13D or combinations thereof. In certain embodiments, the mutated KRAS peptides comprise one or more of SEQ ID NOs: 1-7 or combinations thereof. In certain embodiments, the peptide vaccine further comprises an immunostimulant. In certain embodiments, the immunostimulant comprises polyinosinic:polycytidylic acid (poly(I:C), poly-ICLC, derivatives thereof, or combinations thereof.

In certain aspects, a composition comprises six peptides corresponding to mKRAS G12C, G12V, G12D, G12A, G12R, G13D and an immunostimulant. In certain embodiments, the composition comprises one more of SEQ ID NOs: 1-7 or combinations thereof. In certain embodiments, the immunostimulant comprises polyinosinic:polycytidylic acid (poly(I:C), poly-ICLC, derivatives thereof, or combinations thereof.

In certain aspects, a method of preventing or treating cancer comprising administering to a subject in need thereof, an immunogenic composition or vaccine comprising a plurality of peptides comprising one or more mutations corresponding to an oncogene.

In certain aspects, a method of inducing an immune response to a neoantigen in a subject in need thereof, comprises administering a vaccine comprising one or a plurality of peptides wherein the peptide(s) comprise one or more mutations which induce an immune response. In certain embodiments, the peptide(s) comprise at least 10 amino acid residues. In certain embodiments, the peptide(s) comprise at least 15 amino acid residues. In certain embodiments, the peptide(s) comprise at least 20 amino acid residues. In certain embodiments, the peptide(s) comprise at least 21 amino acid residues. In certain embodiments, the peptide(s) comprise an amino acid sequence of a tumor-associated neoantigen. In certain embodiments, the peptide(s) induce a T cell response. In certain embodiments, the method further comprises administering an adjuvant or immunostimulant. In certain embodiments, the vaccine further comprises administering one or more therapeutic agents, radiation therapy or combinations thereof.

In certain aspects, a method for manufacturing a vaccine, the method comprises steps of: a) detecting a mutation corresponding to a tumor-associated neoantigen, b) preparing one or a plurality of peptides comprising one or more tumor-associated neoantigen mutations; c) assaying for a T cell response to each of the peptides to identify immunogenic mutations and d) manufacturing a vaccine comprising one or more peptides or polypeptides comprising one or more immunogenic mutations. In certain embodiments, the mutations are detected by partial or complete sequencing of a genome, exome, or transcriptome of one or more cells from a subject. In certain embodiments, the one or plurality of peptides or polypeptides comprises one or more immunogenic mutations.

In further aspects, methods for treating a subject suffering from or susceptible to a cancer are provided, including a KRAS-associated cancer. The methods in general comprise administering an effective amount of an immunogenic composition or vaccine as disclosed herein to a subject in need thereto, such as a subject including a human subject identified as suffering from or susceptible to a KRAS-associated cancer, such as a cancer involving or associated with a mutated KRAS gene, or a cancer having abnormal or upregulated KRAS expression.

In embodiments, methods for treating a subject suffering from or susceptible to lung cancer (including non-small lung cancer (NSCLC)), pancreatic cancer and/or colon cancer are provided. The methods in general comprise administering an effective amount of an immunogenic composition or vaccine as disclosed herein to a subject in need thereto, such as a subject including a human subject identified as suffering from or susceptible to lung cancer (including non-small lung cancer (NSCLC)), pancreatic cancer and/or colon cancer. The subject may have for example a resected lung cancer, pancreatic cancer and/or colon cancer

In embodiments, methods for treating a subject suffering from or susceptible to pancreatic cancer, including adenocarcinoma. The subject may have for example a resected pancreatic cancer.

In the present therapeutic methods, an immunogenic composition or vaccine as disclosed herein. suitably may be administered in combination or conjunction with one or more other chemotherapeutic agents.

### Definitions

The terminology used herein is for the purpose of describing particular embodiments only and is not intended to be limiting of the disclosure. Unless specifically defined otherwise, all technical and scientific terms used herein shall be taken to have the same meaning as commonly understood by one of ordinary skill in the art (e.g., in cell culture, molecular genetics, and biochemistry).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

An “adaptive immune response” is an immune response in response to confrontation with an antigen or immunogen, where the immune response is specific for antigenic determinants of the antigen/immunogen-examples of adaptive immune responses are induction of antigen specific antibody production or antigen specific induction/activation of T helper lymphocytes or cytotoxic lymphocytes.

As used herein, an “adjuvant” refers to a substance that enhances the body's immune response to an antigen or a vaccine and may be added to the formulation that includes the immunizing agent. Adjuvants provide enhanced immune response even after administration of only a single dose of the vaccine. Adjuvants may include, for example, aluminum hydroxide and aluminum phosphate, saponins e.g., Quil A, QS-21 (Cambridge Biotech Inc., Cambridge Mass.), GPI-0100 (Galenica Pharmaceuticals, Inc., Birmingham, Ala.), non-metabolizable oil, mineral and/or plant/vegetable and/or animal oils, polymers, carbomers, surfactants, natural organic compounds, plant extracts, carbohydrates, cholesterol, lipids, water-in-oil emulsion, oil-in-water emulsion, water-in-oil-in-water emulsion, HRA-3 (acrylic acid saccharide cross-linked polymer), HRA-3 with cottonseed oil (CSO), or an acrylic acid polyol cross-linked polymer. The emulsion can be based in particular on light liquid paraffin oil (European Pharmacopeia type); isoprenoid oil such as squalane or squalene; oil resulting from the oligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers comprise nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the PLURONIC™ brand products, especially L121. See Hunter et al., The Theory and Practical Application of Adjuvants (Ed. Stewart-Tull, D. E. S.) John Wiley and Sons, NY, pp 51-94 (1995) and Todd et al., Vaccine 15:564-570 (1997). In a preferred embodiment the adjuvant is at a concentration of about 0.01 to about 50%, at a concentration of about 2% to 30%, at a concentration of about 5% to about 25%, at a concentration of about 7% to about 22%, and at a concentration of about 10% to about 20% by volume of the final product. Examples of suitable adjuvants are described in U.S. Patent Application Publication No. US2004/0213817 A1. “Adjuvanted” refers to a composition that incorporates or is combined with an adjuvant.

“Antigen presenting cells” (APC) are cells which present peptide fragments of protein antigens in association with MHC molecules on their cell surface. Some APCs may activate antigen specific T cells. Professional antigen-presenting cells are very efficient at internalizing antigen, either by phagocytosis or by receptor-mediated endocytosis, and then displaying a fragment of the antigen, bound to a class II MHC molecule, on their membrane. The T cell recognizes and interacts with the antigen-class II MHC molecule complex on the membrane of the antigen-presenting cell. An additional co-stimulatory signal is then produced by the antigen-presenting cell, leading to activation of the T cell. The expression of co-stimulatory molecules is a defining feature of professional antigen-presenting cells. The main types of professional antigen-presenting cells are dendritic cells, which have the broadest range of antigen presentation, and are probably the most important antigen-presenting cells, macrophages, B-cells, and certain activated epithelial cells. Dendritic cells (DCs) are leukocyte populations that present antigens captured in peripheral tissues to T cells via both MHC class II and I antigen presentation pathways. It is well known that dendritic cells are potent inducers of immune responses and the activation of these cells is a critical step for the induction of antitumoral immunity. Dendritic cells are conveniently categorized as “immature” and “mature” cells, which can be used as a simple way to discriminate between two well characterized phenotypes. However, this nomenclature should not be construed to exclude all possible intermediate stages of differentiation. Immature dendritic cells are characterized as antigen presenting cells with a high capacity for antigen uptake and processing, which correlates with the high expression of Fcy receptor and mannose receptor. The mature phenotype is typically characterized by a lower expression of these markers, but a high expression of cell surface molecules responsible for T cell activation such as class I and class II MHC, adhesion molecules (e.g. CD54 and CD11) and costimulatory molecules (e.g., CD40, CD80, CD86 and 4-1 BB). Dendritic cell maturation is referred to as the status of dendritic cell activation at which such antigen-presenting dendritic cells lead to T cell priming, while presentation by immature dendritic cells results in tolerance. Dendritic cell maturation is chiefly caused by biomolecules with microbial features detected by innate receptors (bacterial DNA, viral RNA, endotoxin, etc.), pro-inflammatory cytokines (TNF, IL-1, IFNs), ligation of CD40 on the dendritic cell surface by CD40L, and substances released from cells undergoing stressful cell death. The dendritic cells can be derived by culturing bone marrow cells in vitro with cytokines, such as granulocyte-macrophage colony-stimulating factor (GM-CSF) and tumor necrosis factor alpha. Non-professional antigen-presenting cells do not constitutively express the MHC class II proteins required for interaction with naive T cells; these are expressed only upon stimulation of the non-professional antigen-presenting cells by certain cytokines such as IFNγ. Antigen presenting cells can be loaded with MHC class I presented peptides by transducing the cells with nucleic acid, preferably RNA, encoding a peptide or polypeptide comprising the peptide to be presented, e.g. a nucleic acid encoding an antigen or polypeptide used for vaccination.

“Affinity” or “binding affinity” is often measured by equilibrium dissociation constant (K_{D}). A molecule is not (substantially) capable of binding to a target if it has no significant affinity for said target and does not bind significantly to said target in standard assays.

As used herein, the term “agent” refers to any molecule, compound, nucleic acid, nucleic acid based moiety, antibody, antibody-based molecule, protein, protein-based molecule and/or substance for use in the prevention, treatment, management and/or diagnosis of cancer.

The term “amino acid residue,” as used herein, encompasses both naturally-occurring amino acids and non-naturally-occurring amino acids. Examples of non-naturally occurring amino acids include, but are not limited to, D-amino acids (i.e. an amino acid of an opposite chirality to the naturally-occurring form), N-a-methyl amino acids, C-a-methyl amino acids, b-methyl amino acids and D- or L-b-amino acids. Other non-naturally occurring amino acids include, for example, b-alanine (b-Ala), norleucine (Nle), norvaline (Nva), homoarginine (Har), 4-aminobutyric acid (g-Abu), 2-aminoisobutyric acid (Aib), 6-aminohexanoic acid (e-Ahx), ornithine (orn), sarcosine, a-amino isobutyric acid, 3-aminopropionic acid, 2,3-diaminopropionic acid (2,3-diaP), D- or L-phenylglycine, D-(trifluoromethyl)-phenylalanine, and D-p-fluorophenylalanine.

“Antigen processing” or “processing” refers to the degradation of a peptide, polypeptide or protein into procession products, which are fragments of the peptide, polypeptide or protein (e.g., the degradation of a polypeptide into peptides) and the association of one or more of these fragments (e.g., via binding) with MHC molecules for presentation by antigen presenting cells, to specific T cells.

The term “assessing” and “evaluating” are used interchangeably to refer to any form of measurement, and includes determining if an element is present or not. The terms “determining,” “measuring,” “assessing,” and “assaying” are used interchangeably and include both quantitative and qualitative determinations. Assessing can be relative or absolute. “Assessing the presence of” includes determining the amount of something present, as well as determining whether it is present or absent.

The term “biological sample” encompasses a variety of sample types obtained from an organism and can be used in a diagnostic or monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

The term “cancer” includes, but is not limited to, solid cancer and blood borne cancer. The term “cancer” refers to disease of tissues or organs, including but not limited to, cancers of the bladder, bone, blood, brain, breast, cervix, chest, colon, endometrium, esophagus, eye, head, kidney, liver, lymph nodes, lung, mouth, neck, ovaries, pancreas, prostate, rectum, skin, stomach, testis, throat, and uterus. Specific cancers include, but are not limited to, advanced malignancy, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, rectal adenocarcinoma, colorectal cancer, including stage 3 and stage 4 colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waldenstrom's macroglobulinemia, smoldering myeloma, indolent myeloma, fallopian tube cancer, androgen independent prostate cancer, androgen dependent stage IV non metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma.

As used herein, the term “cancer therapy” refers to a therapy useful in treating cancer. Examples of anti-cancer therapeutic agents include, but are not limited to, e.g., surgery, chemotherapeutic agents, immunotherapy, growth inhibitory agents, cytotoxic agents, agents used in radiation therapy, anti-angiogenesis agents, apoptotic agents, anti-tubulin agents, and other agents to treat cancer, such as anti-HER-2 antibodies (e.g., HERCEPTIN™), anti-CD20 antibodies, an epidermal growth factor receptor (EGFR) antagonist (e.g., a tyrosine kinase inhibitor), HER1/EGFR inhibitor (e.g., erlotinib (TARCEVA™)), platelet derived growth factor inhibitors (e.g., GLEEVEC™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also contemplated for use with the methods described herein.

A “chemotherapeutic agent” is a chemical compound useful in the treatment of cancer. Examples of chemotherapeutic agents include nivolumab, ipilimumab, Erlotinib (TARCEVA™, Genentech/OSI Pharm.), Bortezomib (VELCADE™, Millennium Pharm.), Fulvestrant (FASLODEX™, Astrazeneca), Sutent (SU11248, Pfizer), Letrozole (FEMARA™, Novartis), Imatinib mesylate (GLEEVEC™, Novartis), PTK787/ZK 222584 (Novartis), Oxaliplatin (Eloxatin™, Sanofi), 5-FU (5-fluorouracil), Leucovorin, Rapamycin (Sirolimus, RAPAMUNE™, Wyeth), Lapatinib (GSK572016, GlaxoSmithKline), Lonafarnib (SCH 66336), Sorafenib (BAY43-9006, Bayer Labs.), and Gefitinib (IRESSA™, Astrazeneca), AG1478, AG1571 (SU 5271; Sugen), alkylating agents such as Thiotepa and CYTOXAN™ cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analogue topotecan); bryostatin; callystatin; CC-1065 (including its adozcicsin, carzcicsin and bizcicsin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γ1 and calicheamicin omega 1 (Angew Chem. Intl. Ed. Engl. (1994)33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, anthramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN™ doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubcrcidin, ubenimcx, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacytidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK™ polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosinc; arabinoside (“Ara-C”); cyclophosphamide; thiotcpa; taxoids, e.g., TAXOL™ paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE™ doxetaxel (Rhone-Poulenc Rorer, Antony, France); chloranbucil; GEMZAR™ gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE™ vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above.

Also included in this definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX™ (tamoxifen)), raloxifene, droloxifene, 4-hydroxytamoxifen, trioxifene, keoxifene, LY117018, onapristone, and FARESTON™ (toremifene); (ii) aromatase inhibitors that inhibit the enzyme aromatase, which regulates estrogen production in the adrenal glands, such as, for example, 4(5)-imidazoles, aminoglutethimide, MEGASE™ (megestrol acetate), AROMASIN™ (exemestane), formestanie, fadrozole, RIVISOR™ (vorozole), FEMARA™ (letrozole), and ARIMIDEX™ (anastrozole); (iii) anti-androgens such as flutamide, nilutamide, bicalutamide, leuprolide, and goserelin; as well as troxacitabine (a 1,3-dioxolane nucleoside cytosine analog); (iv) aromatase inhibitors; (v) protein kinase inhibitors; (vi) lipid kinase inhibitors; (vii) antisense oligonucleotides, particularly those which inhibit expression of genes in signaling pathways implicated in aberrant cell proliferation, such as, for example, PKC-alpha, Ralf and H-Ras; (viii) ribozymes such as a VEGF expression inhibitor (e.g., ANGIOZYME™ (ribozyme)) and a HER2 expression inhibitor; (ix) vaccines such as gene therapy vaccines, for example, ALLOVECTIN™ vaccine, LEUVECTIN™ vaccine, and VAXID™ vaccine; PROLEUKIN™ rIL-2; LURTOTECAN™ topoisomerase 1 inhibitor; ABARELIX™ rmRH; (x) anti-angiogenic agents such as bevacizumab (AVASTIN™, Genentech); and (xi) pharmaceutically acceptable salts, acids or derivatives of any of the above.

The term “combination therapy”, as used herein, refers to those situations in which two or more different agents are administered in overlapping regimens so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered just followed by one or more additional agents. In the separate administration protocol, two or more agents may be administered a few minutes apart, or a few hours apart, or a few days apart.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

“Diagnosis” as used herein generally includes determination of a subject's susceptibility to a disease or disorder, determination as to whether a subject is presently affected by a disease or disorder, prognosis of a subject affected by a disease or disorder (e.g., identification of pre-metastatic or metastatic cancerous states, stages of cancer, or responsiveness of cancer to therapy), and use of therametrics (e.g., monitoring a subject's condition to provide information as to the effect or efficacy of therapy).

As used herein, the term “effective amount” refers to the amount of a therapy that is sufficient to result in therapeutic benefit to a patient with cancer. In one embodiment, the effective amount is administered to a patient that has been diagnosed with cancer. The effective amount can result in the prevention of the development, recurrence, or onset of cancer and one or more symptoms thereof, to enhance or improve the efficacy of another therapy, reduce the severity, the duration of cancer, ameliorate one or more symptoms of cancer, prevent the advancement of cancer, cause regression of cancer, and/or enhance or improve the therapeutic effect(s) of another therapy. “Effective amount” also refers to the amount of a therapy that is sufficient to result in the prevention of the development, recurrence, or onset of cancer and one or more symptoms thereof, to enhance or improve the prophylactic effect(s) of another therapy, reduce the severity, the duration of cancer, ameliorate one or more symptoms of cancer, prevent the advancement of cancer, cause regression of cancer, and/or enhance or improve the therapeutic effect(s) of another therapy. In an embodiment of the disclosure, the amount of a therapy is effective to achieve one, two, three, or more results following the administration of one, two, three or more therapies: (1) a stabilization, reduction or elimination of the cancer stem cell population; (2) a stabilization, reduction or elimination in the cancer cell population; (3) a stabilization or reduction in the growth of a tumor or neoplasm; (4) an impairment in the formation of a tumor; (5) eradication, removal, or control of primary, regional and/or metastatic cancer; (6) a reduction in mortality; (7) an increase in disease-free, relapse-free, progression-free, and/or overall survival, duration, or rate; (8) an increase in the response rate, the durability of response, or number of patients who respond or are in remission; (9) a decrease in hospitalization rate, (10) a decrease in hospitalization lengths, (11) the size of the tumor is maintained and does not increase or increases by less than 10%, preferably less than 5%, preferably less than 4%, preferably less than 2%, (12) an increase in the number of patients in remission, (13) an increase in the length or duration of remission, (14) a decrease in the recurrence rate of cancer, (15) an increase in the time to recurrence of cancer, and (16) an amelioration of cancer-related symptoms and/or quality of life.

A “growth inhibitory agent” when used herein refers to a compound or composition which inhibits growth of a cell either in vitro or in vivo. Thus, the growth inhibitory agent may be one which significantly reduces the percentage of cells in S phase. Examples of growth inhibitory agents include agents that block cell cycle progression (at a place other than S phase), such as agents that induce Gl arrest and M-phase arrest. Classical M-phase blockers include the vincas (e.g., vincristine and vinblastine), taxanes, and topoisomerase II inhibitors such as doxorubicin, epirubicin, daunorubicin, etoposide, and bleomycin. The agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (WB Saunders: Philadelphia, 1995), especially p. 13. The taxanes (paclitaxel and docetaxel) are anticancer drugs both derived from the yew tree. Docetaxel (TAXOTERE™, Rhone-Poulenc Rorer), derived from the European yew, is a semisynthetic analogue of paclitaxel (TAXOL™, Bristol-Myers Squibb). Paclitaxel and docetaxel promote the assembly of microtubules from tubulin dimers and stabilize microtubules by preventing depolymerization, which results in the inhibition of mitosis in cells.

A “hapten” is a small molecule, which can neither induce or elicit an immune response, but if conjugated to an immunogenic carrier, antibodies or TCRs that recognize the hapten can be induced upon confrontation of the immune system with the hapten carrier conjugate.

The term “human leukocyte antigens” or “HLA”, refers to proteins (antigens) found on the surface of white blood cells and other tissues that are used to match donor and patient. For instances, a patient and potential donor can have their white blood cells tested for such HLA antigens as HLA-A, B and DR. Each individual has two sets of these antigens, one set inherited from each parent. An important aspect of the HLA gene system is its polymorphism. Each gene, MHC class I (A, B and C) and MHC class II (DP, DQ and DR) exists in different alleles. HLA alleles are designated by numbers and subscripts. For example, two unrelated individuals can carry class I HLA-B, genes B5, and Bw41, respectively. Large panels of specific antibodies or nucleic acid reagents are used to type HLA haplotypes of individuals, using leukocytes that express class I and class II molecules. The genes most important for HLA typing are the six MHC Class I and Class II proteins, two alleles for each of HLA-A; HLA-B and HLA-DR. The HLA genes are clustered in a “super-locus” present on chromosome position 6p21, which encodes the six classical transplantation HLA genes and at least 132 protein coding genes that have important roles in the regulation of the immune system as well as some other fundamental molecular and cellular processes. The complete locus measures roughly 3.6 Mb, with at least 224 gene loci. One effect of this clustering is that “haplotypes”, i.e. the set of alleles present on a single chromosome, which is inherited from one parent, tend to be inherited as a group. The set of alleles inherited from each parent forms a haplotype, in which some alleles tend to be associated together.

“An immunogenic carrier” is a molecule or moiety to which an immunogen or a hapten can be coupled in order to enhance or enable the elicitation of an immune response against the immunogen/hapten. Immunogenic carriers are in classical cases relatively large molecules (such as tetanus toxoid, KLH, diphtheria toxoid etc.) which can be fused or conjugated to an immunogen/hapten, which is not sufficiently immunogenic in its own right-typically, the immunogenic carrier is capable of eliciting a strong T-helper lymphocyte response against the combined substance constituted by the immunogen and the immunogenic carrier, and this in turn provides for improved responses against the immungon by B-lymphocytes and cytotoxic lymphocytes. More recently, the large carrier molecules have to a certain extent been substituted by so-called promiscuous T-helper epitopes, i.e. shorter peptides that are recognized by a large fraction of HLA haplotypes in a population, and which elicit T-helper lymphocyte responses.

An “immunogen” is a substance of matter which is capable of inducing an adaptive immune response in a host, whose immune system is confronted with the immunogen. As such, immunogens are a subset of the larger genus “antigens”, which are substances that can be recognized specifically by the immune system (e.g. when bound by antibodies or, alternatively, when fragments of the are antigens bound to MHC molecules are being recognized by T-cell receptors) but which are not necessarily capable of inducing immunity-an antigen is, however, always capable of eliciting immunity, meaning that a host that has an established memory immunity against the antigen will mount a specific immune response against the antigen.

An “immunostimulant” or “immunostimulators” are substances (drugs and nutrients) that stimulate the immune system by inducing activation or increasing activity of any of its components. The term “immunostimulant” may also include an adjuvant. One notable example is the granulocyte macrophage colony-stimulating factor. There are two main categories of immunostimulants: Specific immunostimulants which provide antigenic specificity in immune response, such as vaccines or any antigen. Non-specific immunostimulants act irrespective of antigenic specificity to augment immune response of other antigen or stimulate components of the immune system without antigenic specificity, such as adjuvants and non-specific immunostimulators. Classes of immunostimulants include bacterial vaccines, colony stimulating factors, interferons, interleukins, therapeutic vaccines, vaccine combinations and viral vaccines.

The term “immune effector cell,” as used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NK-T) cells, mast cells, and myeloic-derived phagocytes. “Immune effector function or immune effector response,” as that term is used herein, refers to function or response, e.g., of an immune effector cell, that enhances or promotes an immune attack of a target cell. For example, an immune effector function or response refers a property of a T or NK cell that promotes killing or the inhibition of growth or proliferation, of a target cell. In the case of a T cell, primary stimulation and co-stimulation are examples of immune effector function or response.

“Inducing an immune response” may mean that there was no immune response before induction, but it may also mean that there was a certain level of immune response before induction and after induction said immune response is enhanced. Thus, “inducing an immune response” also includes “enhancing an immune response”. Preferably, after inducing an immune response in a subject, the subject is protected from developing a disease such as a cancer disease or the disease condition is ameliorated by inducing an immune response. For example, an immune response against a tumor-expressed antigen may be induced in a patient having a cancer disease or in a subject being at risk of developing a cancer disease. Inducing an immune response in this case may mean that the disease condition of the subject is ameliorated, that the subject does not develop metastases, or that the subject being at risk of developing a cancer disease does not develop a cancer disease.

“Major histocompatibility complex antigens” (“MHC”, also called “human leukocyte antigens”, HLA) are protein molecules expressed on the surface of cells that confer a unique antigenic identity to these cells. MHC/HLA antigens are target molecules that are recognized by T-cells and natural killer (NK) cells as being derived from the same source of hematopoietic stem cells as the immune effector cells (“self”) or as being derived from another source of hematopoietic reconstituting cells (“non-self”). Two main classes of HLA antigens are recognized: HLA class I and HLA class II. HLA class I antigens (A, B, and C in humans) render each cell recognizable as “self,” whereas HLA class II antigens (DR, DP, and DQ in humans) are involved in reactions between lymphocytes and antigen presenting cells.

The term “neoantigen” relates to a peptide or protein including one or more amino acid modifications compared to the parental peptide or protein. For example, the neoantigen may be a tumor-associated neoantigen, wherein the term “tumor-associated neoantigen” includes a peptide or protein including amino acid modifications due to tumor-specific mutations.

As used herein, “neoplasia” means a disease state of a human or an animal in which there are cells and/or tissues which proliferate abnormally. Neoplastic conditions include, but are not limited to, cancers, sarcomas, tumors, leukemias, lymphomas, and the like. A neoplastic condition refers to the disease state associated with the neoplasia. Colon cancer (e.g., colorectal cancer), lung cancer and ovarian cancer are examples (non-limiting) of a neoplastic condition.

As used in this specification and the appended claims, the term “or” is generally employed in its sense including “and/or” unless the content clearly dictates otherwise.

The terms “patient” or “individual” or “subject” are used interchangeably herein, and refers to a mammalian subject to be treated, with human patients being preferred. In some cases, the methods of the disclosure find use in experimental animals, in veterinary application, and in the development of animal models for disease, including, but not limited to, rodents including mice, rats, and hamsters, and primates.

As used herein, unless otherwise indicated, the terms “peptide”, “polypeptide” or “protein” are used interchangeably herein, and refer to a polymer of amino acids of varying sizes. These terms do not connote a specific length of a polymer of amino acids. Thus, for example, the terms oligopeptide, protein, and enzyme are included within the definition of polypeptide or peptide, whether produced using recombinant techniques, chemical or enzymatic synthesis, or be naturally occurring. This term also includes polypeptides that have been modified or derivatized, such as by glycosylation, acetylation, phosphorylation, and the like.

The term “pharmaceutically acceptable” refers to approved or approvable by a regulatory agency of the Federal or a state government or listed in the U.S. Pharmacopeia or other generally recognized pharmacopeia for use in animals, including humans.

A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

A “pharmaceutically acceptable salt” as recited herein may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH2)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts for the pooled tumor specific neoantigens provided herein, including those listed by Remington's Pharmaceutical Sciences, 17th ed., Mack Publishing Company, Easton, Pa., p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

As used herein, the terms “prevent,” “preventing” and “prevention” in the context of the administration of a therapy to a subject refer to the prevention or inhibition of the recurrence, onset, and/or development of a cancer or a symptom thereof in a subject resulting from the administration of a therapy (e.g., a prophylactic agent), or a combination of therapies (e.g., a combination of prophylactic agents). In some embodiments, such terms refer to one, two, three, or more results following the administration of one or more therapies: (1) a stabilization, reduction or elimination in the cancer cell population, (2) an increase in response rate, (3) an increase in the length or duration of remission, (4) a decrease in the recurrence rate of cancer, (5) an increase in the time to recurrence of cancer, (6) an increase in the disease-free, relapse-free, progression-free, and/or overall survival of the patient, and (7) an amelioration of cancer-related symptoms and/or quality of life.

A “protective, adaptive immune response” is an antigen-specific immune response induced in a subject as a reaction to immunization (artificial or natural) with an antigen, where the immune response is capable of protecting the subject against subsequent challenges with the antigen or a pathology-related agent that includes the antigen. Typically, prophylactic vaccination aims at establishing a protective adaptive immune response against one or several pathogens.

“Stimulation of the immune system” means that a substance or composition of matter exhibits a general, non-specific immunostimulatory effect. A number of adjuvants and putative adjuvants (such as certain cytokines) share the ability to stimulate the immune system. The result of using an immunostimulating agent is an increased “alertness” of the immune system meaning that simultaneous or subsequent immunization with an immunogen induces a significantly more effective immune response compared to isolated use of the immunogen.

The terms “T cell” and “T lymphocyte” are used interchangeably herein and include T helper cells (CD4⁺T cells) and cytotoxic T cells (CTLs, CD8⁺T cells) which comprise cytolytic T cells. Among the sub-types and subpopulations of T cells and/or of CD4⁺ and/or of CD8⁺ T cells are naïve T (T_{N}) cells, effector T cells (T_{EFF}), memory T cells and sub-types thereof, such as stem cell memory T (T_{SCMX} central memory T (T_{CM} effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as T_{H}1 cells, T_{H}2 cells, T_{H}3 cells, T_{H}17 cells, T_{H}9 cells, T_{H}22 cells, follicular helper T cells, alpha/beta T cells, and delta/gamma T cells.

T cells belong to a group of white blood cells known as lymphocytes, and play a central role in cell-mediated immunity. They can be distinguished from other lymphocyte types, such as B cells and natural killer cells by the presence of a special receptor on their cell surface called T cell receptor (TCR). The thymus is the principal organ responsible for the maturation of T cells. Several different subsets of T cells have been discovered, each with a distinct function. T helper cells assist other white blood cells in immunologic processes, including maturation of B cells into plasma cells and activation of cytotoxic T cells and macrophages, among other functions. These cells are also known as CD4⁺T cells because they express the CD4 protein on their surface. Helper T cells become activated when they are presented with peptide antigens by MHC class II molecules that are expressed on the surface of antigen presenting cells (APCs). Once activated, they divide rapidly and secrete small proteins called cytokines that regulate or assist in the active immune response. Cytotoxic T cells destroy virally infected cells and tumor cells, and are also implicated in transplant rejection. These cells are also known as CD8⁺T cells since they express the CD8 glycoprotein at their surface. These cells recognize their targets by binding to antigen associated with MHC class I, which is present on the surface of nearly every cell of the body.

A majority of T cells have a T cell receptor (TCR) existing as a complex of several proteins. The actual T cell receptor is composed of two separate peptide chains, which are produced from the independent T cell receptor alpha and beta (TCRα and TCRβ) genes and are called α- and β-TCR chains. γδ T cells (gamma delta T cells) represent a small subset of T cells that possess a distinct T cell receptor (TCR) on their surface. However, in γδ T cells, the TCR is made up of one γ-chain and one δ-chain. This group of T cells is much less common (2% of total T cells) than the αβ T cells. The first signal in activation of T cells is provided by binding of the T cell receptor to a short peptide presented by the MHC on another cell. This ensures that only a T cell with a TCR specific to that peptide is activated. The partner cell is usually an antigen presenting cell such as a professional antigen presenting cell, usually a dendritic cell in the case of naive responses, although B cells and macrophages can be important APCs.

Cytotoxic T lymphocytes may be generated in vivo by incorporation of an antigen or a peptide fragment thereof into antigen-presenting cells in vivo. The antigen or a peptide fragment thereof may be represented as protein, as DNA (e.g. within a vector) or as RNA. The antigen may be processed to produce a peptide partner for the MHC molecule, while a fragment thereof may be presented without the need for further processing. The latter is the case in particular, if these can bind to MHC molecules. In general, administration to a patient by intradermal injection is possible. However, injection may also be carried out intranodally into a lymph node (Maloy et al. (2001), Proc Natl Acad Sci USA 98:3299-303). The resulting cells present the complex of interest and are recognized by autologous cytotoxic T lymphocytes which then propagate.

Specific activation of CD4⁺ or CD8⁺ T cells may be detected in a variety of ways. Methods for detecting specific T cell activation include detecting the proliferation of T cells, the production of cytokines (e.g., lymphokines), or the generation of cytolytic activity. For CD4⁺ T cells, a preferred method for detecting specific T cell activation is the detection of the proliferation of T cells. For CD8⁺ T cells, a preferred method for detecting specific T cell activation is the detection of the generation of cytolytic activity.

A “T cell epitope” relates to a portion or fragment of an antigen which is capable of stimulating an immune response, such as a cellular response against the antigen or cells characterized by expression of the antigen and by presentation of the antigen such as diseased cells, in particular cancer cells. A T cell epitope is capable of stimulating a cellular response against a cell characterized by presentation of an antigen with class I MHC and is capable of stimulating an antigen-responsive cytotoxic T-lymphocyte (CTL). A T cell epitope may be present in a vaccine as a part of a larger entity such as a vaccine sequence and/or a polypeptide comprising more than one T cell epitope. The presented peptide or T cell epitope is produced following suitable processing.

A “T-helper lymphocyte response” is an immune response elicited on the basis of a peptide, which is able to bind to an MHC class II molecule (e.g. an HLA class II molecule) in an antigen-presenting cell and which stimulates T-helper lymphocytes in an animal species as a consequence of T-cell receptor recognition of the complex between the peptide and the MHC Class II molecule. Assays for determining the effectiveness of a T-cell response can be measured by various assays known in the art, for example mixed lymphocyte reaction; ELISA, flow cytometry, immunohistochemistry, etc.

As defined herein, a “therapeutically effective” amount of a compound or agent (i.e., an effective dosage) means an amount sufficient to produce a therapeutically (e.g., clinically) desirable result. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the disclosure can include a single treatment or a series of treatments.

As used herein, the term “treatment” or “treating” refers to clinical intervention designed to alter the natural course of the individual or cell being treated during the course of clinical pathology, e.g., cancer or tumor immunity. Desirable effects of treatment include decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, delaying the progression of the disease, and/or prolonging survival of individuals. Efficacy of the cancer treatment can be monitored by known methods to those of ordinary skill in the art, including a decrease in size of a palpable tumor, etc., as appropriate for the specific cancer being treated.

As used herein, the terms “treat” and “prevent” are not intended to be absolute terms. In various embodiments, treatment can refer to a 10%, 20%, 30%, 40%, 50%, 60%, 70%, 80%, 90%, or 100% reduction in the severity of an established disease, condition, or symptom of the disease or condition. In embodiments, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. In embodiments, references to decreasing, reducing, or inhibiting include a change of 10%, 20%, 30%, 40%, 50%, 60%, 70%, 80%, 90% or greater as compared to a control level and such terms can include but do not necessarily include complete elimination. In embodiments, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

“Tumor,” as used herein, refers to all neoplastic cell growth and proliferation, whether malignant or benign, and all pre-cancerous and cancerous cells and tissues. The terms “cancer”, “cancerous”, “cell proliferative disorder”, “proliferative disorder” and “tumor” are not mutually exclusive as referred to herein.

As used herein, an “unnatural amino acid,” “non-natural”, “modified amino acid” or “chemically modified amino acid” refers to any amino acid, modified amino acid, or amino acid analogue other than the twenty genetically encoded alpha-amino acids. Unnatural amino acids have side chain groups that distinguish them from the natural amino acids, although unnatural amino acids can be naturally occurring compounds other than the twenty proteinogenic alpha-amino acids. In addition to side chain groups that distinguish them from the natural amino acids, unnatural amino acids may have an extended backbone such as beta-amino acids.

Non-limiting examples of non-natural amino acids include selenocysteine, pyrrolysine, homocysteine, an O-methyl-L-tyrosine, an L-3-(2-naphthyl) alanine, a 3-methyl-phenylalanine, an O-4-allyl-L-tyrosine, a 4-propyl-L-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, an isopropyl-L-phenylalanine, an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or any combination thereof; an amino acid with a photoactivatable cross-linker; a spin-labeled amino acid; a fluorescent amino acid; an amino acid with a novel functional group; an amino acid that covalently or noncovalently interacts with another molecule; a metal binding amino acid; a metal-containing amino acid; a radioactive amino acid; a photocaged and/or photoisomerizable amino acid; a biotin or biotin-analogue containing amino acid; a glycosylated or carbohydrate modified amino acid; a keto containing amino acid; amino acids comprising polyethylene glycol or polyether; a heavy atom substituted amino acid; a chemically cleavable or photocleavable amino acid; an amino acid with an elongated side chain; an amino acid containing a toxic group; a sugar substituted amino acid, e.g., a sugar substituted serine or the like; a carbon-linked sugar-containing amino acid; a redox-active amino acid; an α-hydroxy containing acid; an amino thio acid containing amino acid; an α,αdisubstituted amino acid; a β-amino acid; and a cyclic amino acid other than proline.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Any compositions or methods provided herein can be combined with one or more of any of the other compositions and methods provided herein.

### BRIEF DESCRIPTION OF THE DRAWINGS

FIG. 1A is a schematic representation of a dosing schema for peptide vaccine and immune checkpoint inhibitors.

FIG. 1B are graphs demonstrating ELISpot data from PBMCs from the first two PDAC patients vaccinated with KRAS vaccine.

FIGS. 2A-2K show immunogenicity data testing mutant KRAS peptide vaccine in patients with resected PDAC.

FIGS. 3A-3H show further immunogenicity data testing mutant KRAS peptide vaccine in patients with resected PDAC.

FIG. 4A shows a schedule for vaccination to a patient, and FIGS. 4B-4D show that mKRAS peptide vaccine induces activated and polyfunctional mKRAS-specific CD4 and CD8 T cell responses.

FIG. 5A shows heatmap of immune markers detected by CyTOF and FIG. 5B shows frequency of KRAS-responsive CD4 central memory (CM) and effector memory (EM) T cells or CD8 effector memory (EM) and effector (Eff) at pretreatment (“PreTX”), peak response (“Peak”), and last timepoint on trial (“Last”).

FIG. 6A shows an exemplary trial design and FIGS. 6B-6G show preliminary immune results from prevention vaccine trial.

### DETAILED DESCRIPTION

The present disclosure is related to tumor vaccines for the prevention and/or treatment of very early cancers. Specifically, a vaccine was developed comprising long peptides corresponding to an oncogene—mutated KRAS—that is expressed in the majority of pancreas cancers and is present in the earliest stages of pancreatic cancer development, and is also present on many other cancers. Mutated KRAS is a driver mutation and is critical to the progression from early pre-cancerous cells to invasive cancer. The KRAS vaccine, comprises six long peptides (21 mers) corresponding to the six most common KRAS mutations, teaches the immune system to recognize and kill any cell that expresses mutated KRAS at the earliest stages of cancer development. This targeted approach leads to the induction of activated, mutant-KRAS specific CD4⁺ and CD8⁺ T cells that are capable of killing early cancerous cells. The overall goal of this vaccine is to intercept the progression of early pre-cancer lesions to cancer beginning at the earliest pre-malignant stages. The preclinical data obtained and described in the examples section supports vaccinating against neoantigens or tumor-specific proteins is more likely to work in a pre-malignant environment prior to the development of a significant immunosuppressive tumor microenvironment.

An exemplary amino acid sequence of a human KRAS proto-oncogene is MTEYKLVVVGAGGVGKSALTIQLIQNHFVDEYDPTIEGVDDAFYTLVREIRKHKEK MSKDGKKKKKKSKTKCVIM (UniProt/Swiss-Prot P01116; SEQ ID NO: 1).

In certain embodiments, the KRAS peptides disclosed herein comprise amino acid mutations in SEQ ID NO: 1. In certain embodiments, SEQ ID NO: 1 comprises one or a plurality of amino acid mutations comprising: G12C, G12V, G12D, G12A, G12R, G13D or combinations thereof. In certain embodiments, KRAS peptides comprise a glycine substituted with cysteine at amino acid position 12 of SEQ ID NO: 1, or a glycine substituted with valine at amino acid position 12 of SEQ ID NO: 1, or a glycine substituted with aspartic acid at amino acid position 12 of SEQ ID NO: 1, or a glycine substituted with alanine at amino acid position 12 of SEQ ID NO: 1, or a glycine substituted with arginine at amino acid position 12 of SEQ ID NO: 1, or a glycine substituted with aspartic acid at amino acid position 13 of SEQ ID NO: 1.

In certain embodiments, a KRAS peptide comprises:

G12C:
(SEQ ID NO: 2)
YKLVVVGACGVGKSALTIQLI,
G12V:
(SEQ ID NO: 3)
YKLVVVGAVGVGKSALTIQLI,
G12D:
(SEQ ID NO: 4)
YKLVVVGADGVGKSALTIQLI,
G12A:
(SEQ ID NO: 5)
YKLVVVGAAGVGKSALTIQLI,
G13D:
(SEQ ID NO: 6)
YKLVVVGAGDVGKSALTIQLI,
G12R:
(SEQ ID NO: 7)
YKLVVVGARGVGKSALTIQLI.

In certain embodiments, the peptides of the disclosure comprises a KRAS peptide having at least two or more mutations. Mutations include deletions, substitutions, reversions to a genomic sequence, insertions. In certain embodiments, the KRAS peptides comprise one or more modified or unnatural amino acids, synthetic or derivatives of amino acids, e.g. glycosylated. In certain embodiments, the KRAS peptide comprises at least a 60%, 70%, 75%, 80%, 85%, 86%, 87%, 88%, 89%, 90%, 91%, 92%, 93%, 94%, 95%, 96%, 97%, 98%, or 99%, sequence identity to SEQ ID NO: 1. In certain embodiments, the KRAS peptide comprises at least a 75%, 80%, 85%, 86%, 87%, 88%, 89%, 90%, 91%, 92%, 93%, 94%, 95%, 96%, 97%, 98%, or 99%, sequence identity to SEQ ID NO: 1. In certain embodiments, the KRAS peptide comprises at least a 90%, 91%, 92%, 93%, 94%, 95%, 96%, 97%, 98%, or 99%, sequence identity to SEQ ID NO: 1. In certain embodiments, the KRAS peptide comprises at least a 95%, 96%, 97%, 98%, or 99%, sequence identity to SEQ ID NO: 1. In certain embodiments, the KRAS peptide comprises SEQ ID NO: 1 or fragments thereof.

In certain embodiments, the KRAS peptide comprises at least a 60%, 70%, 75%, 80%, 85%, 86%, 87%, 88%, 89%, 90%, 91%, 92%, 93%, 94%, 95%, 96%, 97%, 98%, or 99%, sequence identity to SEQ ID NO: 2. In certain embodiments, the KRAS peptide comprises at least a 75%, 80%, 85%, 86%, 87%, 88%, 89%, 90%, 91%, 92%, 93%, 94%, 95%, 96%, 97%, 98%, or 99%, sequence identity to SEQ ID NO: 2. In certain embodiments, the KRAS peptide comprises at least a 90%, 91%, 92%, 93%, 94%, 95%, 96%, 97%, 98%, or 99%, sequence identity to SEQ ID NO: 2. In certain embodiments, the KRAS peptide comprises at least a 95%, 96%, 97%, 98%, or 99%, sequence identity to SEQ ID NO: 2. In certain embodiments, the KRAS peptide comprises SEQ ID NO: 2 or fragments thereof.

In certain embodiments, the KRAS peptide comprises at least a 60%, 70%, 75%, 80%, 85%, 86%, 87%, 88%, 89%, 90%, 91%, 92%, 93%, 94%, 95%, 96%, 97%, 98%, or 99%, sequence identity to SEQ ID NO: 3. In certain embodiments, the KRAS peptide comprises at least a 75%, 80%, 85%, 86%, 87%, 88%, 89%, 90%, 91%, 92%, 93%, 94%, 95%, 96%, 97%, 98%, or 99%, sequence identity to SEQ ID NO: 3. In certain embodiments, the KRAS peptide comprises at least a 90%, 91%, 92%, 93%, 94%, 95%, 96%, 97%, 98%, or 99%, sequence identity to SEQ ID NO: 3. In certain embodiments, the KRAS peptide comprises at least a 95%, 96%, 97%, 98%, or 99%, sequence identity to SEQ ID NO: 3. In certain embodiments, the KRAS peptide comprises SEQ ID NO: 3 or fragments thereof.

In certain embodiments, the KRAS peptide comprises at least a 60%, 70%, 75%, 80%, 85%, 86%, 87%, 88%, 89%, 90%, 91%, 92%, 93%, 94%, 95%, 96%, 97%, 98%, or 99%, sequence identity to SEQ ID NO: 4. In certain embodiments, the KRAS peptide comprises at least a 75%, 80%, 85%, 86%, 87%, 88%, 89%, 90%, 91%, 92%, 93%, 94%, 95%, 96%, 97%, 98%, or 99%, sequence identity to SEQ ID NO: 4. In certain embodiments, the KRAS peptide comprises at least a 90%, 91%, 92%, 93%, 94%, 95%, 96%, 97%, 98%, or 99%, sequence identity to SEQ ID NO: 4. In certain embodiments, the KRAS peptide comprises at least a 95%, 96%, 97%, 98%, or 99%, sequence identity to SEQ ID NO: 4. In certain embodiments, the KRAS peptide comprises SEQ ID NO: 4 or fragments thereof.

In certain embodiments, the KRAS peptide comprises at least a 60%, 70%, 75%, 80%, 85%, 86%, 87%, 88%, 89%, 90%, 91%, 92%, 93%, 94%, 95%, 96%, 97%, 98%, or 99%, sequence identity to SEQ ID NO: 5. In certain embodiments, the KRAS peptide comprises at least a 75%, 80%, 85%, 86%, 87%, 88%, 89%, 90%, 91%, 92%, 93%, 94%, 95%, 96%, 97%, 98%, or 99%, sequence identity to SEQ ID NO: 5. In certain embodiments, the KRAS peptide comprises at least a 90%, 91%, 92%, 93%, 94%, 95%, 96%, 97%, 98%, or 99%, sequence identity to SEQ ID NO: 5. In certain embodiments, the KRAS peptide comprises at least a 95%, 96%, 97%, 98%, or 99%, sequence identity to SEQ ID NO: 5. In certain embodiments, the KRAS peptide comprises SEQ ID NO: 5 or fragments thereof.

In certain embodiments, the KRAS peptide comprises at least a 60%, 70%, 75%, 80%, 85%, 86%, 87%, 88%, 89%, 90%, 91%, 92%, 93%, 94%, 95%, 96%, 97%, 98%, or 99%, sequence identity to SEQ ID NO: 6. In certain embodiments, the KRAS peptide comprises at least a 75%, 80%, 85%, 86%, 87%, 88%, 89%, 90%, 91%, 92%, 93%, 94%, 95%, 96%, 97%, 98%, or 99%, sequence identity to SEQ ID NO: 6. In certain embodiments, the KRAS peptide comprises at least a 90%, 91%, 92%, 93%, 94%, 95%, 96%, 97%, 98%, or 99%, sequence identity to SEQ ID NO: 6. In certain embodiments, the KRAS peptide comprises at least a 95%, 96%, 97%, 98%, or 99%, sequence identity to SEQ ID NO: 6. In certain embodiments, the KRAS peptide comprises SEQ ID NO: 6 or fragments thereof.

In certain embodiments, the KRAS peptide comprises at least a 60%, 70%, 75%, 80%, 85%, 86%, 87%, 88%, 89%, 90%, 91%, 92%, 93%, 94%, 95%, 96%, 97%, 98%, or 99%, sequence identity to SEQ ID NO: 7. In certain embodiments, the KRAS peptide comprises at least a 75%, 80%, 85%, 86%, 87%, 88%, 89%, 90%, 91%, 92%, 93%, 94%, 95%, 96%, 97%, 98%, or 99%, sequence identity to SEQ ID NO: 7. In certain embodiments, the KRAS peptide comprises at least a 90%, 91%, 92%, 93%, 94%, 95%, 96%, 97%, 98%, or 99%, sequence identity to SEQ ID NO: 7. In certain embodiments, the KRAS peptide comprises at least a 95%, 96%, 97%, 98%, or 99%, sequence identity to SEQ ID NO: 7. In certain embodiments, the KRAS peptide comprises SEQ ID NO: 7 or fragments thereof.

In certain embodiments, a vaccine formulation comprises one or more KRAS peptides comprising SEQ ID NOs: 1, 2, 3, 4, 5, 6, 7 or combinations thereof.

Accession numbers for KRAS gene: GeneID:3845; HGNC:HGNC:6407; MIM:190070HGNC:6407, incorporated herein by reference in their entirety.

### Neoantigens

Neoantigens are a class of HLA-bound peptides that arise from cancer-specific mutations. Specifically, as provided herein a “neoantigen” is intended to mean a unique, new antigen to a specific cancer, tumor, or cell thereof, which arises as a consequence of the accumulation of random mutations from aberrant DNA replication and/or repair in the cancer, tumor, or cell thereof. Effective anti-cancer immunity in humans has been associated with the presence of T-cells directed at cancer neoantigens. Thus, identification of immunogenic neoantigens represent a promising target for anti-cancer vaccines.

Genome instability and mutations are a hallmark of cancer. When the mutations are not inherited and occur at some time after conception, the mutations are termed somatic mutations. The somatic mutations can be present in a specific population of cells. For example, when the mutation leads to a malignant growth the somatic mutation can be unique to the cancer cells. When the somatic mutations occur in the coding regions of the genome, the mutations have the potential to generate neoantigens. In certain embodiments, the mutations are cancer specific somatic mutations in a tumor specimen of a cancer patient which may be determined by identifying sequence differences between the genome, exome and/or transcriptome of a tumor specimen and the genome, exome and/or transcriptome of a non-tumor specimen.

The neoantigens are important from an immunology standpoint, because the neoantigens are antigens to which the immune system has not been previously exposed. As a result, neoantigens can represent a vulnerability for cancer cells if they become recognized by the immune system as foreign. The successful recognition of a neoantigen on a cancer cell can trigger an immune response to specifically target and destroy the cancer cells. Therefore, neoantigens are considered important targets for cancer immunotherapy because of their immunogenicity and lack of expression in normal tissues. Neoantigens can be identified in various ways, such as, for example, genetic means, computer-based analysis, machine-learning and the like.

GENETIC MEANS: Neoantigens can be identified by determining the nucleotide sequence of a cancer cell and comparing it to a reference sequence. In some embodiments, the reference sequence can be a normal cell from the same subject. For example, a biopsy can be collected from a subject and sequencing can be performed on the cancer cells or tumor specimen and the normal cells adjacent to the cancer. A tumor specimen relates to any sample such as a bodily sample derived from a patient containing or being expected of containing tumor or cancer cells. The bodily sample may be any tissue sample such as blood, a tissue sample obtained from the primary tumor or from tumor metastases or any other sample containing tumor or cancer cells. Preferably, a bodily sample is blood and cancer specific somatic mutations or sequence differences are determined in one or more circulating tumor cells (CTCs) contained in the blood. In another embodiment, a tumor specimen relates to one or more isolated tumor or cancer cells such as circulating tumor cells (CTCs) or a sample containing one or more isolated tumor or cancer cells such as circulating tumor cells (CTCs). The reference sequence can be from a publicly available sequence of a healthy subject or a consensus sequence of multiple healthy subjects. For example, the reference sequence can be derived by determining the nucleotide sequence in two or more healthy subjects and generating a consensus sequence based on perfect homology between the subjects.

DNA and/or RNA can be prepared using, for example, nucleic acid purification methods. Subsequently, the nucleic acids can be sequenced using, for example, Sanger sequencing, Next-generation sequencing, or related methods capable of identifying the precise sequence of nucleotides in the DNA and/or RNA. For example, sequencing can be performed by whole genome sequencing (WGS) of a subject's DNA, or specific subsets of the genome can be sequenced, such as whole exon sequencing (WES). In addition, gene expression analysis by RNA sequencing (RNA-seq), that is transcriptome sequencing, or microarrays can be used to predict candidate neoantigens derived from the somatic mutations detected by WES.

Differences between mutated peptide sequence in cancer cells and the wild-type peptide sequence can identify potential neoantigen peptides. For example, the nucleotide sequence of the exonic DNA or the messenger RNA from a cancer cell can be translated into an encoded peptide sequence. Exemplary wild-type peptide sequences can be obtained by sequencing the nucleotide sequence of the exonic DNA or the messenger RNA from a normal tissue and translating the nucleotide sequence into an encoded peptide sequence. Additional wild-type peptide sequences can be used as a reference, and need to be from a paired sample from a normal tissue. For example, a consensus wild-type peptide sequence obtained from patient's blood or two or more healthy subjects can serve as the wild-type peptide sequence. Peptide sequences originating from the cancer cells that contain at least one amino acid that differs from a corresponding wild-type peptide sequence can be considered a neoantigen peptide.

The sequencing can be performed on various samples collected from a subject having or suspected of having cancer. Exemplary samples include a tumor biopsy, a benign sample, or a blood sample with circulating tumor DNA. The sequencing can be performed on a single cell, or a pool of cells. In silico tools can be used to identify mutations and the encoding neoantigen peptides.

Any suitable sequencing method can be used according to the disclosure for determining mutations, Next Generation Sequencing (NGS) technologies being preferred. Third Generation Sequencing methods might substitute for the NGS technology in the future to speed up the sequencing step of the method. For clarification purposes: the terms “Next Generation Sequencing” or “NGS” in the context of the present disclosure mean all novel high throughput sequencing technologies which, in contrast to the “conventional” sequencing methodology known as Sanger chemistry, read nucleic acid templates randomly in parallel along the entire genome by breaking the entire genome into small pieces. Such NGS technologies (also known as massively parallel sequencing technologies) are able to deliver nucleic acid sequence information of a whole genome, exome, transcriptome (all transcribed sequences of a genome) or methylome (all methylated sequences of a genome) in very short time periods, e.g. within 1-2 weeks or within 1-7 days or within less than 24 hours and allow, in principle, single cell sequencing approaches. Multiple NGS platforms which are commercially available or which are mentioned in the literature can be used in the context of the present disclosure e.g. those described in detail in Zhang et al. 2011: The impact of next-generation sequencing on genomics. J. Genet Genomics38(3), 95-109; or in Voelkerding et al. 2009: Next generation sequencing: From basic research to diagnostics. Clinical chemistry 55, 641-658. Non-limiting examples of such NGS technologies/platforms include:
1) The sequencing-by-synthesis technology known as pyrosequencing implemented e.g. in the GS-FLX 454 Genome Sequencer™ of Roche-associated company 454 Life Sciences (Branford, Conn.), first described in Ronaghi et al. 1998: A sequencing method based on real-time pyrophosphate”. Science 281(5375), 363-365. This technology uses an emulsion PCR in which single-stranded DNA binding beads are encapsulated by vigorous vortexing into aqueous micelles containing PCR reactants surrounded by oil for emulsion PCR amplification. During the pyrosequencing process, light emitted from phosphate molecules during nucleotide incorporation is recorded as the polymerase synthesizes the DNA strand.
2) The sequencing-by-synthesis approaches developed by Solexa (now part of Illumina Inc., San Diego, Calif.) which is based on reversible dye-terminators and implemented e.g. in the Illumina/Solexa Genome Analyzer™ and in the Illumina HiSeq 2000 Genome Analyzer™. In this technology, all four nucleotides are added simultaneously into oligo-primed cluster fragments in flow-cell channels along with DNA polymerase. Bridge amplification extends cluster strands with all four fluorescently labeled nucleotides for sequencing.
3) Sequencing-by-ligation approaches, e.g. implemented in the SOLid™ platform of Applied Biosystems (now Life Technologies Corporation, Carlsbad, Calif.). In this technology, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting bead, each containing only copies of the same DNA molecule, are deposited on a glass slide. As a second example, the Polonator™ G.007 platform of Dover Systems (Salem, N.H.) also employs a sequencing-by-ligation approach by using a randomly arrayed, bead-based, emulsion PCR to amplify DNA fragments for parallel sequencing.
4) Single-molecule sequencing technologies such as e.g. implemented in the PacBio RS system of Pacific Biosciences (Menlo Park, Calif.) or in the HeliScope™ platform of Helicos Biosciences (Cambridge, Mass.). The distinct characteristic of this technology is its ability to sequence single DNA or RNA molecules without amplification, defined as Single-Molecule Real Time (SMRT) DNA sequencing. For example, HeliScope uses a highly sensitive fluorescence detection system to directly detect each nucleotide as it is synthesized. A similar approach based on fluorescence resonance energy transfer (FRET) has been developed from Visigen Biotechnology (Houston, Tex.). Other fluorescence-based single-molecule techniques are from U.S. Genomics (GeneEngine™) and Genovoxx (AnyGene™).
5) Nano-technologies for single-molecule sequencing in which various nanostructures are used which are e.g. arranged on a chip to monitor the movement of a polymerase molecule on a single strand during replication. Non-limiting examples for approaches based on nano-technologies are the GridON™ platform of Oxford Nanopore Technologies (Oxford, UK), the hybridization-assisted nano-pore sequencing (HANS™) platforms developed by Nabsys (Providence, R.I.), and the proprietary ligase-based DNA sequencing platform with DNA nanoball (DNB) technology called combinatorial probe-anchor ligation (cPAL™).
6) Electron microscopy based technologies for single-molecule sequencing, e.g. those developed by LightSpeed Genomics (Sunnyvale, Calif.) and Halcyon Molecular (Redwood City, Calif.)
7) Ion semiconductor sequencing which is based on the detection of hydrogen ions that are released during the polymerisation of DNA. For example, Ion Torrent Systems (San Francisco, Calif.) uses a high-density array of micro-machined wells to perform this biochemical process in a massively parallel way. Each well holds a different DNA template. Beneath the wells is an ion-sensitive layer and beneath that a proprietary Ion sensor.

DNA and RNA preparations serve as starting material for NGS. Such nucleic acids can be easily obtained from samples such as biological material, e.g. from fresh, flash-frozen or formalin-fixed paraffin embedded tumor tissues (FFPE) or from freshly isolated cells or from CTCs which are present in the peripheral blood of patients. Normal non-mutated genomic DNA or RNA can be extracted from normal, somatic tissue, however germline cells are preferred in the context of the present disclosure. Germline DNA or RNA may be extracted from peripheral blood mononuclear cells (PBMCs) in patients with non-hematological malignancies. Although nucleic acids extracted from FFPE tissues or freshly isolated single cells are highly fragmented, they are suitable for NGS applications.

Several targeted NGS methods for exome sequencing are described in the literature (for review see e.g. Teer and Mullikin 2010: Human Mol Genet 19 (2), R145-51), all of which can be used in conjunction with the present disclosure. Many of these methods (described e.g. as genome capture, genome partitioning, genome enrichment etc.) use hybridization techniques and include array-based (e.g. Hodges et al. 2007: Nat. Genet. 39, 1522-1527) and liquid-based (e.g. Choi et al. 2009: Proc. Natl. Acad. Sci USA 106, 19096-19101) hybridization approaches. Commercial kits for DNA sample preparation and subsequent exome capture are also available: for example, Illumina Inc. (San Diego, Calif.) offers the TruSeq™ DNA Sample Preparation Kit and the Exome Enrichment Kit TruSeq™ Exome Enrichment Kit.

In order to reduce the number of false positive findings in detecting cancer specific somatic mutations or sequence differences when comparing e.g. the sequence of a tumor sample to the sequence of a reference sample such as the sequence of a germ line sample it is preferred to determine the sequence in replicates of one or both of these sample types. Thus, it is preferred that the sequence of a reference sample such as the sequence of a germ line sample is determined twice, three times or more. Alternatively or additionally, the sequence of a tumor sample is determined twice, three times or more. It may also be possible to determine the sequence of a reference sample such as the sequence of a germ line sample and/or the sequence of a tumor sample more than once by determining at least once the sequence in genomic DNA and determining at least once the sequence in RNA of said reference sample and/or of said tumor sample. For example, by determining the embodiments between replicates of a reference sample such as a germ line sample the expected rate of false positive (FDR) somatic mutations as a statistical quantity can be estimated. Technical repeats of a sample should generate identical results and any detected mutation in this “same vs. same comparison” is a false positive. In particular, to determine the false discovery rate for somatic mutation detection in a tumor sample relative to a reference sample, a technical repeat of the reference sample can be used as a reference to estimate the number of false positives. Furthermore, various quality related metrics (e.g. coverage or SNP quality) may be combined into a single quality score using a machine learning approach. For a given somatic variation all other embodiments with an exceeding quality score may be counted, which enables a ranking of all embodiments in a dataset.

In the context of the present disclosure, the term “RNA” relates to a molecule which comprises at least one ribonucleotide residue and preferably being entirely or substantially composed of ribonucleotide residues. “Ribonucleotide” relates to a nucleotide with a hydroxyl group at the 2′-position of a β-D-ribofuranosyl group. The term “RNA” comprises double-stranded RNA, single-stranded RNA, isolated RNA such as partially or completely purified RNA, essentially pure RNA, synthetic RNA, and recombinantly generated RNA such as modified RNA which differs from naturally occurring RNA by addition, deletion, substitution and/or alteration of one or more nucleotides. Such alterations can include addition of non-nucleotide material, such as to the end(s) of a RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs or analogs of naturally-occurring RNA.

The abundance of a neoantigen peptide can also influence the efficacy of a neoantigen peptide as a target for an anti-cancer vaccine. For example, a neoantigen peptide that is expressed at low levels on a cancer cell can be unlikely to be encountered by a T-cell, even if the neoantigen has been presented to effector T-cells and memory T-cells have been formed. Conversely, a neoantigen peptide that is expressed at relatively high levels can have a higher probability of encountering T-cells surveillance. The abundance of the neoantigen peptide can be determined by sequencing methods, such as those described above. Abundance can be determined by gene expression, as well as allelic expression. Therefore, in some embodiments, the current subject matter provided herein further includes determining the abundance of the neoantigen peptide using exome, transcriptome, or whole genome sequencing.

In some embodiments, the neoantigen peptides with the highest expression can be prioritized as being the best candidates for an anticancer vaccine. In some embodiments, the expression can be calculated from sequencing RNA. In other embodiments, the expression can be determined by sequencing the expression of specific alleles that contain somatic mutations.

COMPUTER-BASED METHODS: Computational approaches can be employed to facilitate identification of immunogenic tumor neoantigens using machine learning algorithms. For example, a computational immunogenic neoantigen prediction pipeline can be employed that combines machine learning predictors trained on (i) Mass Spectrometry (MS) data of peptides eluted from a MHC molecule, (ii) T-cell activation/interaction assay output data, and/or (iii) prediction of MHC-binding affinity. The integration of the output from the machine learning models into a single composite score can enable the precise identification of personalized cancer vaccine candidates.

In some embodiments, the neoantigen peptides can be between 9 to 25 amino acids in length. In certain embodiments, the neoantigen peptides that serve as input to a machine learning algorithm are twenty one amino acids in length. Consequently, in some embodiments, the input to a machine learning algorithm can comprise all of the various sequences of 21 amino acids that originate from a larger peptide with the same amino acid sequence order. For example, if a neoantigen peptide is 21 amino acids in length, then the input for that specific neoantigen peptide can comprise multiple different possible combinations of the amino acids that can be generated from the same sequence order of amino acids. In certain embodiments, the peptides comprise one or more mutations that correspond to mutations found in oncogenes, such as for example, KRAS.

The neoantigen peptide can be used to generate a naturally processed (NP) antigen predictor score (NP-predictor) score. This can be achieved using a first machine learning model that is trained using data derived from mass spectrometry (MS) of isolated peptides eluted from at least one MHC molecule. High-throughput, high-quality MS experiments have identified a number of naturally processed (NP) antigen peptides from various pathogens, cancer cell lines and human tumor samples. These peptides represent bona fide antigens that are processed by antigen presenting cells and presented on MHC molecules. Furthermore, a large fraction of these peptides is not accurately predicted using MHC-binding prediction algorithms alone.

Identification of the NP peptides eluted from MHC can be performed by immunoprecipitation of MHC molecules followed by peptide elution, purification, and analysis by liquid chromatography-MS/MS. The elution data can include MHC class I and MHC class II data. Further, the data can be generated from human and non-human samples, such as mouse. Peptides identified from elution of MHC molecules represent validated MHC-presenting epitopes that can provide an accurate prediction of peptides that are likely to elicit T-cell receptor interactions. As such, the naturally processed peptides can complement or enhance output information generation from the prediction of MHC-binding affinity.

The data that is used for training the machine learning algorithm for a NP predictor score can be selected and curated from experimental evidence of peptides eluted from MHC molecules. For example, the data can be collected from publicly available experimental results and include the peptide sequence, associated HLA allele type, source protein ID, MS abundance percentage, and predicted or experimentally measured HLA-binding affinity.

The data used for determining the NP predictor score need not be from mono-allelic cells. The data can also be determined from multi-allelic cells. For example, for multi-allelic cells the HLA allele with the strongest binding to the eluted peptide can be designated as the associated allele. Alternatively, a MHC binding affinity prediction model can be used for multi-allelic cells without experimental data on HLA binding affinity. For example, a peptide can be subjected to an MHC binding affinity prediction model, and among the eluted HLA alleles the one that is predicted to have the strongest binding to the eluted peptide can be designated as the associated allele.

In some embodiments, the MS abundance percentage (0-100%) can be incorporated into the machine learning algorithm. For example, a peptide with an abundance of 94.2% can be incorporated as 0.942. Alternatively, a peptide that does not have experimental data on peptide abundance can be treated as 100% and be assigned a value of 1.0. For example, a peptide without abundance data, but detected from the MHC eluted samples, can be set at 1.0. Therefore, any peptide detected from the MHC eluted samples can be given an MS abundance value greater than 0 and less than or equal to 1.0.

The machine learning algorithm for generating an NP-predictor score can also be trained using negative labels. Exemplary negative labels can be generated from random sequences of nine amino acids collected from the same or similar set of source genes used to map the positive human proteome database. For example, the MS-positive peptides and the random sequences can be generated using the human proteome database. A local alignment with the positive MS-identified peptides can be performed to exclude any sequences where more than four amino acid residues were aligned between the positive and the negative peptides. The number of negative decoys can be 1,000 peptides, 5,000 peptides, 10,000 peptides, 50,000 peptides, 100,000 peptides, or 500,000 peptides or more peptides that are not present in the MHC-eluted peptide data as decoys. Such negative labels can be given a label of 0.0 that corresponds with the abundance score of the positive peptides described above.

Various machine learning algorithms can be used to analyze the neoantigen input against the mass-spec identified MHC-eluted data to generate a NP-predictor score. In some embodiments, the first machine learning model is a neural network (NN) that generates a NP-predictor score. In certain embodiments, the neural network is a convolutional neural network, a recurrent neural network, or a deep learning neural network. It is understood that the first learning model can also generate a NP-predictor score using other machine learning algorithms and that the neural network algorithm is intended to be exemplary. For example, in some embodiments, the first machine learning model is a random forest, logistic regression, or an unsupervised clustering model.

An exemplary first machine learning model using a random forest or a logistic regression model can utilize training data input where the NP peptide sequences and the output is a class label of positive vs. negative. Alternatively, an exemplary first machine learning model can use an unsupervised clustering model that includes training data input from NP peptide sequences identified from a list of, for example, specific human HLA types such that the model assigns each sequence to a unique HLA type. The predictor therefore predicts the possibility of one particular sequence being eluted/naturally presented by that specific HLA type.

In other embodiments of neural network models, the model can also be a deep neural network (DNN) with multiple locally and fully connected hidden layers, or a high-order neural network (HONN). For DNN, a Restricted Boltzmann Machine (RBM) can be used to pre-train the neural nodes of input and connecting layers. For HONN, a mean-covariance RBM can be used to pre-train the neural nodes of input and connecting layers.

The affinity that a neoantigen peptide has for an MHC molecule is an additional factor that can influence the immunogenicity of a neoantigen peptide. Although prediction of the binding affinity alone does not accurately predict the efficacy of a peptide in being able to elicit an immune response, the binding of a peptide to an MHC molecule is an integral step in the process of a peptide eliciting an immune response. Therefore, the neoantigen peptide is received by a machine learning model to generate a score predicting affinity of the neoantigen peptide for binding an MHC molecule (MHC binding score). The prediction of peptide-MHC binding can be performed using machine learning algorithms. In some embodiments, the machine learning algorithm for predicting peptide-MHC binding is an artificial neural network. The ANN can be trained for the different MHC alleles, including HLA-A, HLA-B, HLA-C, and HLA-E. In some embodiments the HLA allele is specific to 4-digits, for example HLA-A*01:01. Publicly available prediction tools are available for predicting peptide-MHC binding. In some embodiments, the NetMHC server can be employed for prediction of peptide-MHC class I binding using artificial neural networks (ANNs).

The identification of neoantigen peptides that can be naturally processed and presented on an MHC molecule are important determinants for whether a neoantigen can engage a T-cell and exhibit an immune response. However, antigen processing and an antigen's affinity for MHC binding are not necessarily indicative of T-cell activation. For example, a peptide can be naturally processed, have strong affinity for an MHC molecule, or both, and yet exhibit little to no T-cell activation. Consequently, the identification of an immunogenic neoantigen peptide can be aided further by analyzing peptides that have a functionally validated immune response to a known peptide sequence. Therefore, in some embodiments, the neoantigen peptide is received by a second machine learning model to generate a T-epitope predictor score, where the machine learning model is trained using experimentally characterized peptides recognized by T-cells.

Various parameters can be used to measure a T-cell response. Exemplary experimental evidence of T-cell activation includes secretion of specific cytokines (e.g., interleukins, interferon-gamma, tumor necrosis factor alpha, and granzymes A and B), proliferation of T-cells, functional responses such as cytotoxicity, and qualitative T-cell binding to an antigen presenting cells (APC). In some embodiments, the machine learning model for a T-epitope predictor score is trained using data collected from T-cell assays of experimentally characterized peptides recognized by T-cells. In some embodiments, the T-cell assays can measure cytokine release, cytotoxicity, or qualitative T-cell binding to an APC. Additional evidence can also be considered, and it is understood that the above is not intended to be inclusive of all indicators or T-cell activation.

Markers of T-cell activation can be used to determine immunogenicity of a peptide. One marker of T-cell activation is cytokine release. Many cytokines and their function of the immune response are well defined. An exemplary function of a well-defined cytokine is the ability of interleukin-2 (IL-2) to stimulate growth of T-cells. Similarly, interleukin-4 (IL-4) can stimulate growth, as well as survival, of T-cells. Tumor necrosis factor alpha (TNFα) is another well-defined cytokine secreted by T-cells that can activate macrophages and induce nitric oxide production, which is a powerful cytotoxic chemical. In some embodiments, the T-cell assay measure cytokine release. In some embodiments, the cytokine is selected from the group of interferon gamma (IFNγ), tumor necrosis factor alpha (TNFα), interleukin-2 (IL-2), interleukin-4 (IL-4), interleukin-5 (IL-5), interleukin-6 (IL-6), interleukin-8 (IL-8), interleukin-10 (IL-10), interleukin-17 (IL-17), interleukin-21 (IL-21), interleukin-22 (IL-22), granzyme A, granzyme B, or any combination thereof.

The data for generating the experimentally characterized peptides from cytokine release can be from any assay used to measure cytokine release. For example, the T-cell assay for measuring IFNy release can be an ELISPOT, ELISA, or similar assay that measures cytokine release. Data can be collected from other exemplary assays that measure cytokine release, and it is understood that the examples described above are intended to be exemplary. In addition, it is understood that the experimental data need not include information on all, or any, of the cytokines mentioned above, and that cytokine release is merely one type of assay that can used to characterized a neoantigen peptide that elicits a T-cell response.

Another exemplary T-cell assay from which data characterizing a neoantigen's immunogenicity can be drawn is a qualitative T-cell binding assay. In some embodiments, the qualitative T-cell binding assay data is multimer/tetramer qualitative binding. For example, the machine learning algorithm can analyze data from a flow cytometry assay that provides experimental evidence on cell-cell binding of a T cell epitope:MHC:T-cell receptor (TCR) complex. Another exemplary assay includes a MHC tetramer staining to identify peptides that are recognized by a T-cell. The machine learning algorithm for generating a T-epitope predictor score can incorporate data from additional T-cell assays, including those not described previously, and the assays describe above are intended to be exemplary support of the type of experimental evidence that can be used for training the machine learning model using experimentally characterized peptides recognized by T-cells.

Yet another exemplary T-cell assay that can be used for characterizing a neoantigen's immunogenicity is a T-cell mediated cytotoxicity assay. For example, the assay can measure the directed killing of a target cell by a T-cell through the release of granules containing cytotoxic mediators or through the engagement of death receptors. In certain embodiments, the assay for measuring cytotoxicity is a chromium-51 (⁵¹Cr) release assay. Measurement of cytotoxicity can be performed by a variety of assays, and the examples provided above are understood to be merely exemplary support of the type of experimental evidence that can be used for training the machine learning model using experimentally characterized peptides recognized by T-cells.

The neoantigen peptide sequence used as input for generating the T-epitope predictor score can be of any length. For example, in some embodiments, the neoantigen peptide is less than or equal to 50 amino acids, less than or equal to 40 amino acids, less than or equal to 30 amino acids, less than or equal to 20 amino acids, or less than or equal to 10 amino acids. In some embodiments, the neoantigen peptide is 21 amino acids.

The data collected from the experimentally characterized peptides recognized by T-cells can include binary class labels. For example, a neoantigen peptide that has been experimentally validated to activate T-cells by a T-cell assay, such as any one of the assays describe previously, can be considered “positive” and be assigned a value of 1.0. An alternative example can be a neoantigen peptide that has been characterized to not elicit any activation of T-cells. Such an exemplary peptide would be assigned a value of 0.0. Any assay that measures T-cell activation can be employed, whether or not it has been described previously, and the peptide can be assigned a binary class label according to the experimentally characterized peptides.

The experimentally characterized peptides recognized by T-cells can be collected from experiments performed by the user, by experiments selected from a public database, or both. An exemplary database of experimentally characterized peptides recognized by T-cells is the Immune Epitope Database (IEDB). Therefore, in some embodiments, the experimentally characterized peptides recognized by T-cells are selected from IEDB. However, any database of experimentally characterized peptides can be used for the T-epitope predictor score, and it is understood that IEDB is an exemplary database. In some embodiments, data from a database can be combined with personal data that is not found in the public database. For example, a user can compile data from publications, generate new data, or acquire data that is not found in the database and use it for training the machine learning model. The data used to train the machine learning model for the T-epitope predictor score can be from ex vivo, in vitro, or in vivo data. In certain embodiments, the data is from ex vivo or in vitro restimulation experiments.

The machine learning model for generating the T-epitope predictor can also be configured to consider specific HLA and/or H2 alleles. In certain embodiments, the HLA can be specific to the HLA locus and be selected from the group of HLA-A, HLA-B, HLA-C, HLA-DRB, HLA-DQA, HLA-DQB, HLA-DPA, or HLA-DPB. In some embodiments, the HLA allele can be specific to 4-digits (e.g. HLA-A*02:01). In some embodiments, the H2 alleles can be specific to the mouse H2 locus and be selected from the group of H2-Db or H2-Kb.

The algorithms for the machine learning model used to generate the T-epitope predictor can be selected from the group of a support vector machine, a Bayesian classifier, a random forest model, a logistic regression model, a boosting classifier, or a neural network. Depending on the amount of training data available for certain HLA alleles, specific algorithms may be better suited for a given dataset that relates to a specific HLA allele. For example, a neural network algorithm can be ineffective for a dataset with limited training data.

The selection of a specific machine learning algorithm for a specific HLA can be performed using a set of models, such as boosting regression, random forest, or support vector machine. The models can be trained and tested on the same 3-folds cross-validation dataset. For example, the training data can be split into three subsets (e.g. A, B, and C) and the model can be trained using two of the different subsets (e.g. A and B). The remaining subset (e.g. C) can then be used test the fitness of the algorithm. The process can be repeated using different permutations of the subsets until the model with the best auROC or lowest mean absolute error can be selected for a given HLA type. Finally, the model can then be re-trained using the entire data available unique to that HLA type.

The data collected from the experimentally characterized peptides recognized by T-cells can also be subjected to dimensionality reduction. In some embodiments, the dimensionality reduction includes principal component analysis (PCA), singular value decomposition (SVD), or non-negative matrix factorization (NVF).

The output generated from the NP-predictor score and the T-epitope predictor score can be combined togetherto generate an ImmunoGenScore. The ImmunoGenScore incorporates the likelihood of both MHC-presentation and T-cell receptor (TCR) interaction. ImmunoGenScores can then be compiled into a ranking list of immunogenic neoantigen peptides that are ordered from highest to lowest, with the highest score being ranked first. Similarly, a separate ranking list can be generated for the neoantigen peptides according to their MHC binding score, with the highest score being ranked first. Subsequently, peptides that are the highest in both rankings can be identified as immunogenic neoantigen peptides. For example, in certain embodiments, the top 50% of neoantigen peptides from the ImmunoGenScore ranking list can be selected, and the top 10% of neoantigen peptides from the MHC binding score ranking list can be cross-referenced. Peptides that are common to both can be selected and given a composite score based on their ranking in both lists. The criteria for selecting the top peptides in each list can be adjusted according to the user's preference. For example, the top 50%, 40%, 30%, 20%, 10%, 5%, 1% or any number in-between can be selected from one or both lists. It is understood that more stringent criteria will yield fewer neoantigen peptides being identified. However, it is also understood that the more stringent criteria can more accurately predict neoantigen peptides that are immunogenic. Alternatively, less stringent conditions will yield more neoantigen peptides, and can include more peptides that are not immunogenic.

### Immunostimulants

Immunostimulants known as immunostimulators are attractive substances that activate the immune system of humans and animals for prevention of diseases and improvement of the body's natural resistance to various viral and bacterial infections. These biologically active substances are the products derived from natural sources or synthetically made with different chemical properties and mechanisms of action. In general, immunostimulants induce synthesis of specific antibodies and cytokines for treatment of infectious diseases. Two major groups of immunostimulants contain a) specific immunostimulants acting as antigen for stimulation of immune responses (e.g., vaccines), and b) non-specific immunostimulants without antigenic properties enhancing immune responses to other antigens (e.g., adjuvants and non-specific immunostimulators).

Immunostimulants activate different elements of the immune system in humans and animals. They develop the non-specific immunotherapy and immunoprevention by stimulating the major factors of the immune system including phagocytosis, properdin and complement systems, protective secretory IgA antibodies, α- and γ-interferon release, T- and B-lymphocytes, synthesis of specific antibodies and cytokines, and synthesis of pulmonary surfactant. There are several reasons for using the immunostimulants in the control of various infectious diseases including: a) antibiotic resistance of the bacteria; b) allergic reactions to antibiotics; c) immunosuppressive effects of antibiotics; and d) Poor effects of the antibiotics in viral infections (Sepideh Shahbazi, Azam Bolhassani. “Immunostimulants: Types and Functions”. JoMMID 2016, 4 (3 And 4): 45-51).

Accordingly, in some embodiments an immunostimulant is administered in combination with the peptides embodied herein. In some embodiments, the immunostimulant is administered prior to vaccination, in combination with the vaccines, post-vaccination or as needed and determined by a healthcare worker. In certain aspects the immunostimulant may be a toll-like receptor (TLR) agonist. TLR agonists comprise flagellins from Salmonella enterica or Vibrio cholerae. TLR agonists may be specific for certain TLR classes (i.e., TLR5, TLR7 or TLR9 agonists) and may be presented in any combination or as any modification. Examples of such immune adjuvants are described in WO 2012/021834, the contents of which are incorporated herein by reference.

In some embodiments, an immunostimulant is an agent that does not constitute a specific antigen, but can boost the strength and longevity of an immune response to an antigen. Such immunostimulants may include, but are not limited to stimulators of pattern recognition receptors, such as Toll-like receptors, RIG-1 and NOD-like receptors (NLR), mineral salts, such as alum, alum combined with monphosphoryl lipid (MPL) A of Enterobacteria, such as Escherihia coli, Salmonella minnesota, Salmonella typhimurium, or Shigella flexneri or specifically with MPL™ (ASO4), MPL A of above-mentioned bacteria separately, saponins, such as QS-21, Quil-A, ISCOMs, ISCOMATRIX, emulsions such as MF59, Montanide, ISA 51 and ISA 720, AS02 (QS21+squalene+MPL.), liposomes and liposomal formulations such as AS01, synthesized or specifically prepared microparticles and microcarriers such as bacteria-derived outer membrane vesicles (OMV) of N. gonorrhoeae, Chlamydia trachomatis and others, or chitosan particles, depot-forming agents, such as Pluronic block co-polymers, specifically modified or prepared peptides, such as muramyl dipeptide, aminoalkyl glucosaminide 4-phosphates, such as RC529, or proteins, such as bacterial toxoids or toxin fragments.

In some embodiments, the immunostimulant comprises an agonist for pattern recognition receptors (PRR), including, but not limited to Toll-Like Receptors (TLRs), specifically TLRs 2, 3, 4, 5, 7, 8, 9 and/or combinations thereof. In some embodiments, additional agents comprise agonists for Toll-Like Receptors 3, agonists for Toll-Like Receptors 7 and 8, or agonists for Toll-Like Receptor 9. In some embodiments, the immunostimulants comprise imidazoquinolines; such as R848; adenine derivatives, such as those disclosed in U.S. Pat. No. 6,329,381, U.S. Published Patent Application 2010/0075995, or WO 2010/018132; immunostimulatory DNA; or immunostimulatory RNA. In some embodiments, the immunostimulant may comprise immunostimulatory RNA molecules, such as but not limited to dsRNA, poly I:C, poly I:poly C12U (available as Ampligen™, both poly I:C and poly I:polyC12U being known as TLR3 stimulants), poly-ICLC (such as Hiltonol), and/or those disclosed in F. Heil et al., “Species-Specific Recognition of Single-Stranded RNA via Toll-like Receptor 7 and 8” Science 303(5663), 1526-1529 (2004); J. Vollmer et al., “Immune modulation by chemically modified ribonucleosides and oligoribonucleotides” WO 2008033432 A2; A. Forsbach et al., “Immunostimulatory oligoribonucleotides containing specific sequence motif(s) and targeting the Toll-like receptor 8 pathway” WO 2007062107 A2; E. Uhlmann et al., “Modified oligoribonucleotide analogs with enhanced immunostimulatory activity” U.S. Pat. Appl. Publ. No. 2006/241076; G. Lipford et al., “Immunostimulatory viral RNA oligonucleotides and use for treating cancer and infections” WO 2005097993 A2; G. Lipford et al., “Immunostimulatory G,U-containing oligoribonucleotides, compositions, and screening methods” WO 2003086280 A2. In some embodiments, an additional agent may be a TLR-4 agonist, such as bacterial lipopolysaccharide (LPS), VSV-G, and/or HMGB-1. In some embodiments, additional agents may comprise TLR-5 agonists, such as flagellin, or portions or derivatives thereof, including but not limited to those disclosed in U.S. Pat. Nos. 6,130,082, 6,585,980, and 7,192,725.

In some embodiments, the immunostimulant may be proinflammatory stimuli released from necrotic cells (e.g., urate crystals). In some embodiments, the immunostimulant may be activated components of the complement cascade (e.g., CD21, CD35, etc.). In some embodiments, the immunostimulant may be activated components of immune complexes. Immunostimulants also include complement receptor agonists, such as a molecule that binds to CD21 or CD35. In some embodiments, the complement receptor agonist induces endogenous complement opsonization of the synthetic nanocarrier. In some embodiments, immunostimulants are cytokines, which are small proteins or biological factors (in the range of 5 kD-20 kD) that are released by cells and have specific effects on cell-cell interaction, communication and behavior of other cells. In some embodiments, the cytokine receptor agonist is a small molecule, antibody, fusion protein, or aptamer.

The immunostimulant can be administered to the patient, parenterally, including intramuscular, intraarterial, intrathecal, intradermal, intraperitoneal, intrasplenic, subcutaneous, and intravenous administration, and particularly injected directly into the tumor, for example to at least one tumor nodule. The dose of immunostimulant can be delivered at appropriate intervals, e.g. 1, 2, 3, or more injections at daily, semi-daily, weekly intervals concurrent with, or following vaccination with the peptides embodied herein.

### Combination Therapies

In certain embodiments, a vaccine formulation comprises one or more KRAS peptides comprising SEQ ID NOs: 1, 2, 3, 4, 5, 6, 7 or combinations thereof. In certain embodiments, the peptides embodied herein can be administered in combination with one or more agents. In certain embodiments, a subject who is at risk of developing cancer is administered the peptides embodied herein to prevent the development of cancer. Such individuals can be determined, for example, by genetics, environmental aspects, biomarkers etc. In certain embodiments, the peptides embodied herein can be administered to a subject in the early stages of cancer. In certain embodiments, the peptides embodied herein are administered in combination with one or more agents, radiation therapy, surgery and the like.

In certain embodiments, a method of preventing or treating a subject at risk of or diagnosed as having cancer comprises administering one or more peptides embodied herein in combination with nivolumab and ipilimumab. Nivolumab (OPDIVO, Bristol-Myers Squibb Co.) is an anti-PD-1antibody that promotes the tumor-killing effects of T-cells. Ipilimumab (YERVOY, Bristol-Myers Squibb Co.) is an anti-CTLA-4 antibody that helps strengthen the immune system by promoting the function and growth of T-cells.

In certain embodiments, a subject is diagnosed as having cancer, e.g. early stage cancer. In certain embodiments, the type of cancer is identified and the cancer is treated by various therapeutics, including therapeutics specific for the type of cancer. The cancer treatment can be surgery, adjuvant chemotherapy, neoadjuvant chemotherapy, radiation therapy, hormone therapy, cytotoxic therapy, immunotherapy, adoptive T cell therapy, targeted therapy, or any combinations thereof. The method also can include administering to the mammal a cancer treatment (e.g., surgery, adjuvant chemotherapy, neoadjuvant chemotherapy, radiation therapy, hormone therapy, cytotoxic therapy, immunotherapy, adoptive T cell therapy, targeted therapy, or any combinations thereof). The mammal can be monitored for the presence of cancer after administration of the cancer treatment.

Cancer therapies in general also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, famesyl-protein transferase inhibitors, transplatinum, 5-fluorouracil, vincristine, vinblastine and methotrexate, Temazolomide (an aqueous form of DTIC), or any analog or derivative variant of the foregoing. The combination of chemotherapy with biological therapy is known as biochemotherapy. The chemotherapy may also be administered at low, continuous doses which is known as metronomic chemotherapy.

Yet further combination chemotherapies include, for example, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; carboplatin, procarbazine, plicomycin, gemcitabien, navelbine, farnesyl-protein transferase inhibitors, transplatinum; and pharmaceutically acceptable salts, acids or derivatives of any of the above.

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells as well as genetically engineered variants of these cell types modified to express chimeric antigen receptors.

The immunotherapy may comprise suppression of T regulatory cells (Tregs), myeloid derived suppressor cells (MDSCs) and cancer associated fibroblasts (CAFs). In some embodiments, the immunotherapy is a tumor vaccine (e.g., whole tumor cell vaccines, peptides, and recombinant tumor associated antigen vaccines), or adoptive cellular therapies (ACT) (e.g., T cells, natural killer cells, TILs, and LAK cells). The T cells may be engineered with chimeric antigen receptors (CARs) or T cell receptors (TCRs) to specific tumor antigens. As used herein, a chimeric antigen receptor (or CAR) may refer to any engineered receptor specific for an antigen of interest that, when expressed in a T cell, confers the specificity of the CAR onto the T cell. Once created using standard molecular techniques, a T cell expressing a chimeric antigen receptor may be introduced into a patient, as with a technique such as adoptive cell transfer. In some aspects, the T cells are activated CD4 and/or CD8 T cells in the individual which are characterized by γ-IFN-producing CD4 and/or CD8 T cells and/or enhanced cytolytic activity relative to prior to the administration of the combination. The CD4 and/or CD8 T cells may exhibit increased release of cytokines selected from the group consisting of IFN-γ, TNF-α and interleukins. The CD4 and/or CD8 T cells can be effector memory T cells. In certain embodiments, the CD4 and/or CD8 effector memory T cells are characterized by having the expression of CD44^{high} CD62L^{low}.

The immunotherapy may be a cancer vaccine comprising one or more cancer antigens, in particular a protein or an immunogenic fragment thereof, DNA or RNA encoding said cancer antigen, in particular a protein or an immunogenic fragment thereof, cancer cell lysates, and/or protein preparations from tumor cells. As used herein, a cancer antigen is an antigenic substance present in cancer cells. In principle, any protein produced in a cancer cell that has an abnormal structure due to mutation can act as a cancer antigen. In principle, cancer antigens can be products of mutated Oncogenes and tumor suppressor genes, products of other mutated genes, overexpressed or aberrantly expressed cellular proteins, cancer antigens produced by oncogenic viruses, oncofetal antigens, altered cell surface glycolipids and glycoproteins, or cell type-specific differentiation antigens. Examples of cancer antigens include the abnormal products of ras and p53 genes. Other examples include tissue differentiation antigens, mutant protein antigens, oncogenic viral antigens, cancer-testis antigens and vascular or stromal specific antigens. Tissue differentiation antigens are those that are specific to a certain type of tissue. Mutant protein antigens are likely to be much more specific to cancer cells because normal cells shouldn't contain these proteins. Normal cells will display the normal protein antigen on their MHC molecules, whereas cancer cells will display the mutant version. Some viral proteins are implicated in forming cancer, and some viral antigens are also cancer antigens. Cancer-testis antigens are antigens expressed primarily in the germ cells of the testes, but also in fetal ovaries and the trophoblast. Some cancer cells aberrantly express these proteins and therefore present these antigens, allowing attack by T-cells specific to these antigens. Exemplary antigens of this type are CTAG1 B and MAGEA1 as well as Rindopepimut, a 14-mer intradermal injectable peptide vaccine targeted against epidermal growth factor receptor (EGFR) v111 variant. Rindopepimut is particularly suitable for treating glioblastoma when used in combination with an inhibitor of the CD95/CD95L signaling system as described herein. Also, proteins that are normally produced in very low quantities, but whose production is dramatically increased in cancer cells, may trigger an immune response. An example of such a protein is the enzyme tyrosinase, which is required for melanin production. Normally tyrosinase is produced in minute quantities but its levels are very much elevated in melanoma cells. Oncofetal antigens are another important class of cancer antigens. Examples are alphafetoprotein (AFP) and carcinoembryonic antigen (CEA). These proteins are normally produced in the early stages of embryonic development and disappear by the time the immune system is fully developed. Thus self-tolerance does not develop against these antigens. Abnormal proteins are also produced by cells infected with oncoviruses, e.g. EBV and HPV. Cells infected by these viruses contain latent viral DNA which is transcribed and the resulting protein produces an immune response. A cancer vaccine may include a peptide cancer vaccine, which in some embodiments is a personalized peptide vaccine. In some embodiments. the peptide cancer vaccine is a multivalent long peptide vaccine, a multi-peptide vaccine, a peptide cocktail vaccine, a hybrid peptide vaccine, or a peptide-pulsed dendritic cell vaccine

The immunotherapy may be an antibody, such as part of a polyclonal antibody preparation, or may be a monoclonal antibody. The antibody may be a humanized antibody, a chimeric antibody, an antibody fragment, a bispecific antibody or a single chain antibody. An antibody as disclosed herein includes an antibody fragment, such as, but not limited to, Fab, Fab′ and F(ab′)2, Fd, single-chain Fvs (scFv), single-chain antibodies, disulfide-linked Fvs (sdfv) and fragments including either a VL or VH domain. In some aspects, the antibody or fragment thereof specifically binds epidermal growth factor receptor (EGFR1, Erb-B1), HER2/neu (Erb-B2), CD20, Vascular endothelial growth factor (VEGF), insulin-like growth factor receptor (IGF-1R), TRAIL-receptor, epithelial cell adhesion molecule, carcino-embryonic antigen, Prostate-specific membrane antigen, Mucin-1, CD30, CD33, or CD40.

Examples of monoclonal antibodies include, without limitation, nivolumab, ipilimumab trastuzumab (anti-HER2/neu antibody); Pertuzumab (anti-HER2 mAb); cetuximab (chimeric monoclonal antibody to epidermal growth factor receptor EGFR); panitumumab (anti-EGFR antibody); nimotuzumab (anti-EGFR antibody); Zalutumumab (anti-EGFR mAb); Necitumumab (anti-EGFR mAb); MDX-210 (humanized anti-HER-2 bispecific antibody); MDX-210 (humanized anti-HER-2 bispecific antibody); MDX-447 (humanized anti-EGF receptor bispecific antibody); Rituximab (chimeric murine/human anti-CD20 mAb); Obinutuzumab (anti-CD20 mAb); Ofatumumab (anti-CD20 mAb); Tositumumab-1131 (anti-CD20 mAb); Ibritumomab tiuxetan (anti-CD20 mAb); Bevacizumab (anti-VEGF mAb); Ramucirumab (anti-VEGFR2 mAb); Ranibizumab (anti-VEGF mAb); Aflibercept (extracellular domains of VEGFR1 and VEGFR2 fused to IgG1 Fc); AMG386 (angiopoietin-1 and -2 binding peptide fused to IgG1 Fc); Dalotuzumab (anti-IGF-1R mAb); Gemtuzumab ozogamicin (anti-CD33 mAb); Alemtuzumab (anti-Campath-1/CD52 mAb); Brentuximab vedotin (anti-CD30 mAb); Catumaxomab (bispecific mAb that targets epithelial cell adhesion molecule and CD3); Naptumomab (anti-5T4 mAb); Girentuximab (anti-Carbonic anhydrase ix); or Farletuzumab (anti-folate receptor). Other examples include antibodies such as Panorex™ (17-1A) (murine monoclonal antibody); Panorex (MAb17-1A) (chimeric murine monoclonal antibody); BEC2 (ami-idiotypic mAb, mimics the GD epitope) (with BCG); Oncolym (Lym-1 monoclonal antibody); SMART M195 Ab, humanized 13′ 1 LYM-1 (Oncolym), Ovarex (B43.13, anti-idiotypic mouse mAb); 3622W94 mAb that binds to EGP40 (17-1A) pancarcinoma antigen on adenocarcinomas; Zenapax (SMART Anti-Tac (IL-2 receptor); SMART M195 Ab, humanized Ab, humanized); NovoMAb-G2 (pancarcinoma specific Ab); TNT (chimeric mAb to histone antigens); TNT (chimeric mAb to histone antigens); Gliomab-H (Monoclonals-Humanized Abs); GNI-250 Mab; EMD-72000 (chimeric-EGF antagonist); LymphoCide (humanized IL.L.2 antibody); and MDX-260 bispecific, targets GD-2, ANA Ab, SMART IDIO Ab, SMART ABL 364 Ab or ImmuRAIT-CEA. Further examples of antibodies include Zanulimumab (anti-CD4 mAb), Keliximab (anti-CD4 mAb); Ipilimumab (MDX-101; anti-CTLA-4 mAb); Tremilimumab (anti-CTLA-4 mAb); (Daclizumab (anti-CD25/IL-2R mAb); Basiliximab (anti-CD25/IL-2R mAb); MDX-1106 (anti-PD1 mAb); antibody to GITR; GC1008 (anti-TGF-β antibody); metelimumab/CAT-192 (anti-TGF-β antibody); lerdelimumab/CAT-152 (anti-TGF-β antibody); ID11 (anti-TGF-β antibody); Denosumab (anti-RANKL mAb); BMS-663513 (humanized anti-4-1BB mAb); SGN-40 (humanized anti-CD40 mAb); CP870,893 (human anti-CD40 mAb); Infliximab (chimeric anti-TNF mAb; Adalimumab (human anti-TNF mAb); Certolizumab (humanized Fab anti-TNF); Golimumab (anti-TNF); Etanercept (Extracellular domain of TNFR fused to IgG1 Fc); Belatacept (Extracellular domain of CTLA-4 fused to Fc); Abatacept (Extracellular domain of CTLA-4 fused to Fc); Belimumab (anti-B Lymphocyte stimulator); Muromonab-CD3 (anti-CD3 mAb); Otelixizumab (anti-CD3 mAb); Teplizumab (anti-CD3 mAb); Tocilizumab (anti-IL6R mAb); REGN88 (anti-IL6R mAb); Ustekinumab (anti-IL-12/23 mAb); Briakinumab (anti-IL-12/23 mAb); Natalizumab (anti-α4 integrin); Vedolizumab (anti-α4 B7 integrin mAb); T1 h (anti-CD6 mAb); Epratuzumab (anti-CD22 mAb); Efalizumab (anti-CD11a mAb); and Atacicept (extracellular domain of transmembrane activator and calcium-modulating ligand interactor fused with Fc).

In certain embodiments, the peptides embodied herein can be administered in combination with immune checkpoint modulators. Immune checkpoints refer to inhibitory pathways of the immune system that are responsible for maintaining self-tolerance and modulating the duration and amplitude of physiological immune responses.

Certain cancer cells thrive by taking advantage of immune checkpoint pathways as a major mechanism of immune resistance, particularly with respect to T cells that are specific for tumor antigens. For example, certain cancer cells may overexpress one or more immune checkpoint proteins responsible for inhibiting a cytotoxic T cell response. Thus, immune checkpoint modulators may be administered to overcome the inhibitory signals and permit and/or augment an immune attack against cancer cells. Immune checkpoint modulators may facilitate immune cell responses against cancer cells by decreasing, inhibiting, or abrogating signaling by negative immune response regulators (e.g. CTLA4), or may stimulate or enhance signaling of positive regulators of immune response (e.g. CD28).

Immunotherapy agents targeted to immune checkpoint modulators may be administered to encourage immune attack targeting cancer cells. Immunotherapy agents may be or include antibody agents that target (e.g., are specific for) immune checkpoint modulators. Examples of immunotherapy agents include antibody agents targeting one or more of CTLA-4, PD-1, PD-L1, GITR, OX40, LAG-3, KIR, TIM-3, CD28, CD40; and CD137.

Specific examples of antibody agents may include monoclonal antibodies. Certain monoclonal antibodies targeting immune checkpoint modulators are available. For instance, ipilumimab targets CTLA-4; tremelimumab targets CTLA-4; pembrolizumab targets PD-1, etc.

### Pharmaceutical Compositions

In certain embodiments, a pharmaceutical composition comprises an effective amount of one or more antigenic peptides as described herein (including a pharmaceutically acceptable salt, thereof), optionally in combination with a pharmaceutically acceptable carrier, excipient or additive.

The peptides described herein are suitable for use in a variety of drug delivery systems. Additionally, in order to enhance the in vivo serum half-life of the administered compound, the compositions may be encapsulated, introduced into the lumen of liposomes, prepared as a colloid, or other conventional techniques may be employed which provide an extended serum half-life of the compositions. A variety of methods are available for preparing liposomes, as described in, e.g., Szoka, et al., U.S. Pat. Nos. 4,235,871, 4,501,728 and 4,837,028 each of which is incorporated herein by reference. Furthermore, one may administer the drug in a targeted drug delivery system, for example, in a liposome coated with a tissue-specific antibody. The liposomes will be targeted to and taken up selectively by the organ. The present disclosure also provides pharmaceutical compositions comprising one or more of the compositions described herein. Formulations may be employed in admixtures with conventional excipients, i.e., pharmaceutically acceptable organic or inorganic carrier substances suitable for administration to the wound or treatment site. The pharmaceutical compositions may be sterilized and if desired mixed with auxiliary agents, e.g., lubricants, preservatives, stabilizers, wetting agents, emulsifiers, salts for influencing osmotic pressure buffers, coloring, and/or aromatic substances and the like. They may also be combined where desired with other active agents, e.g., other analgesic agents.

Administration of the compositions of this disclosure may be carried out, for example, by parenteral, by intravenous, intratumoral, subcutaneous, intramuscular, or intraperitoneal injection, or by infusion or by any other acceptable systemic method. Formulations for administration of the compositions include those suitable for rectal, nasal, oral, topical (including buccal and sublingual), vaginal or parenteral (including subcutaneous, intramuscular, intravenous and intradermal) administration. The formulations may conveniently be presented in unit dosage form, e.g. tablets and sustained release capsules, and may be prepared by any methods well known in the art of pharmacy.

As used herein, “additional ingredients” include, but are not limited to, one or more of the following: excipients; surface active agents; dispersing agents; inert diluents; granulating and disintegrating agents; binding agents; lubricating agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the disclosure are known in the art and described, for example in Genaro, ed. (1985, Remington's Pharmaceutical Sciences, Mack Publishing Co., Easton, PA), which is incorporated herein by reference.

The compositions of the disclosure may comprise a preservative from about 0.005% to 2.0% by total weight of the composition. The preservative is used to prevent spoilage in the case of exposure to contaminants in the environment. Examples of preservatives useful in accordance with the disclosure included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

Liquid suspensions may be prepared using conventional methods to achieve suspension the composition of the disclosure in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid.

This disclosure is further illustrated by the following examples which should not be construed as limiting. The contents of all references, patents, and published patent applications cited throughout this application, as well as the figures, are hereby incorporated by reference.

### EXAMPLES

### Example 1: Neoantigen Vaccine

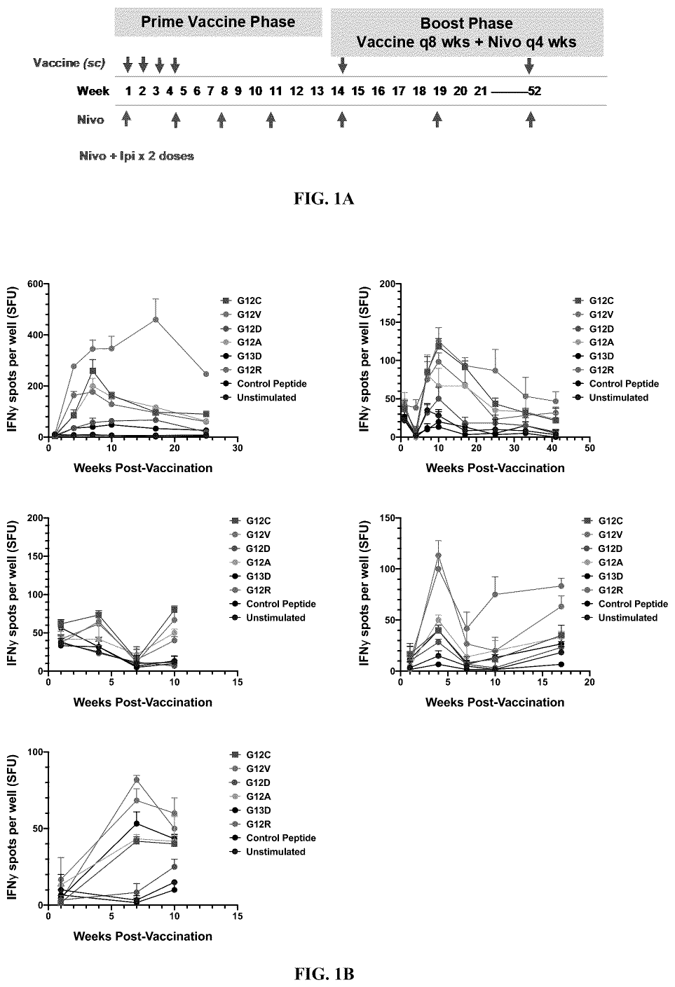

The clinical study conducted, tested the immunogenicity of a mutant KRAS (mKRAS) vaccine in combination with nivolumab and ipilimumab in twelve patients with resected pancreatic cancer who have completed peri-operative chemotherapy with or without radiation treatment (J1994). A pooled long peptide vaccine corresponding to mKRAS G12C (SEQ ID NO: 2), G12V (SEQ ID NO: 3), G12D (SEQ ID NO: 4), G12A (SEQ ID NO: 5), G12R (SEQ ID NO: 7), and G13D (SEQ ID NO: 6) was developed. The vaccine consisted of the six 21mer peptides with the mutated residue at position 9 or 10 admixed with poly-ICLC (Hiltonol). Seven patients were enrolled and vaccinated with resected pancreatic ductal adenocarcinoma (PDAC) thus far. Longitudinal ELISpot data available from the first six PDAC patients demonstrated the induction of robust de novo mKRAS specific T cell responses against many of the peptides included in the vaccine, including the ones expressed in each patient's respective tumor (FIGS. 1A, 1B).

### Example 2: Immunogenicity Data Testing Mutant KRAS Peptide Vaccine in Patients With Resected PDAC

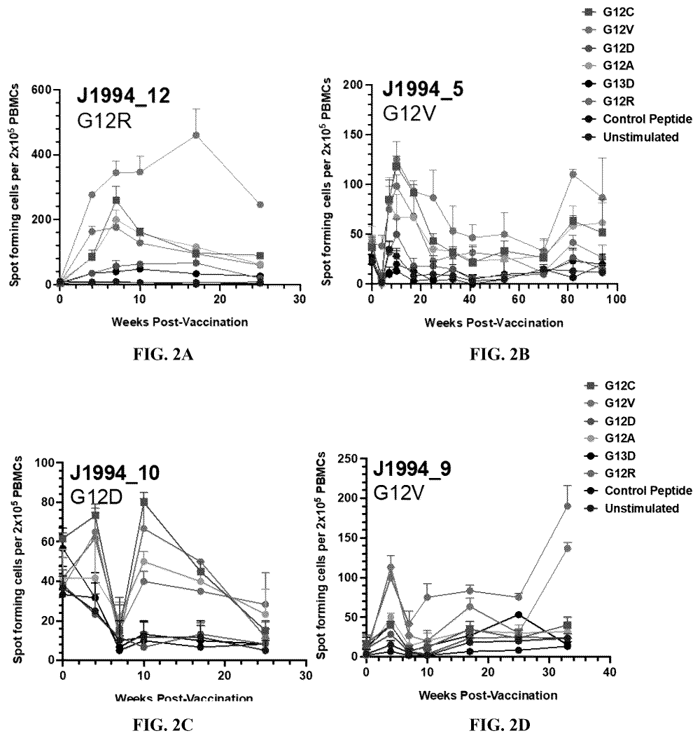

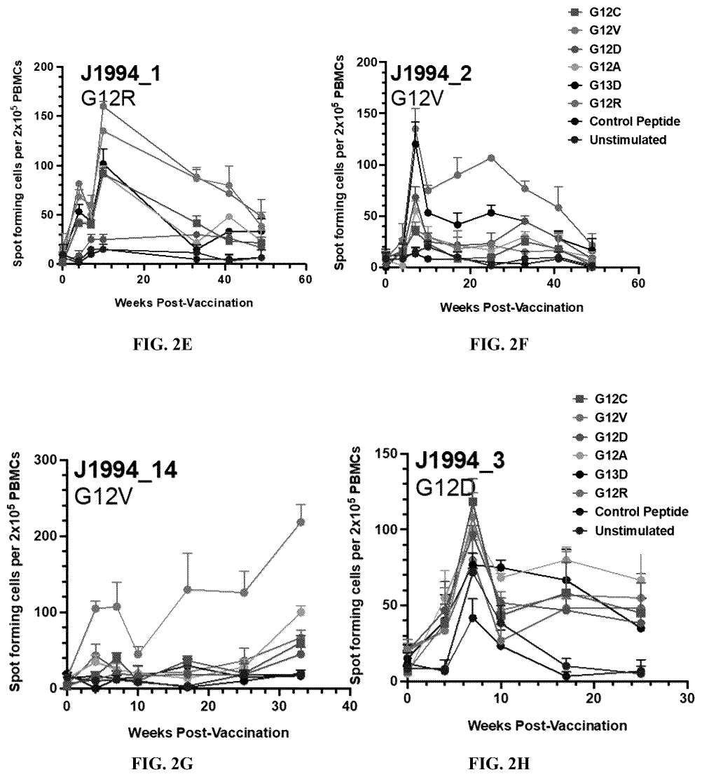

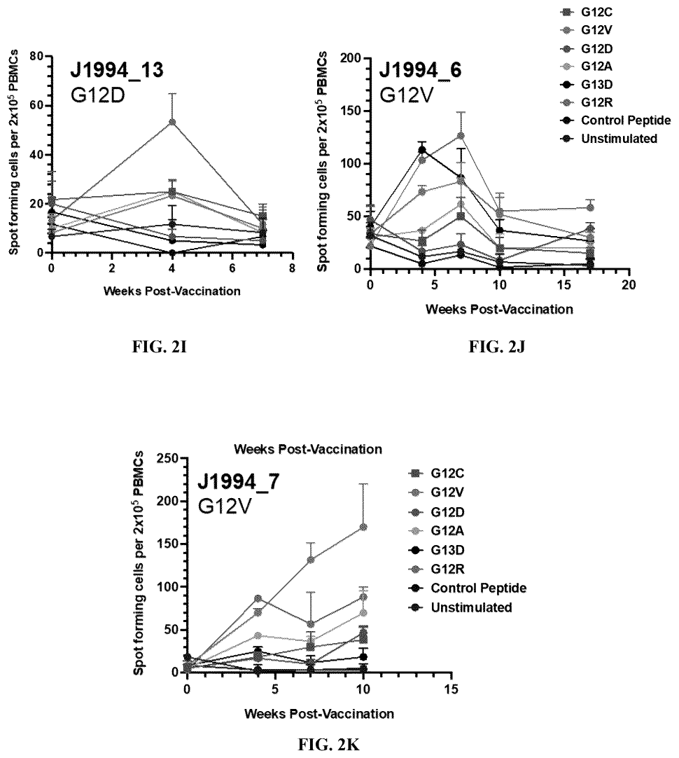

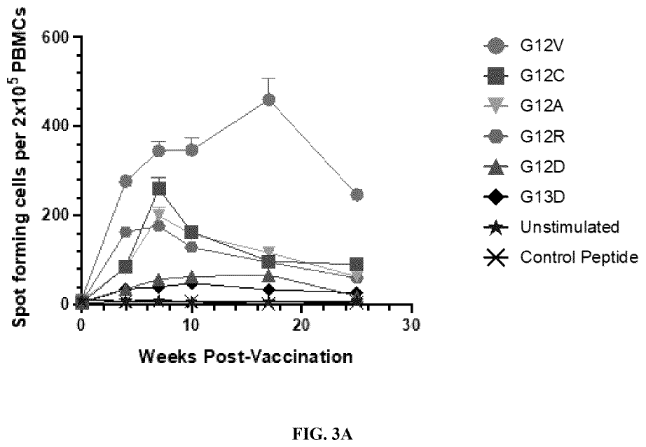

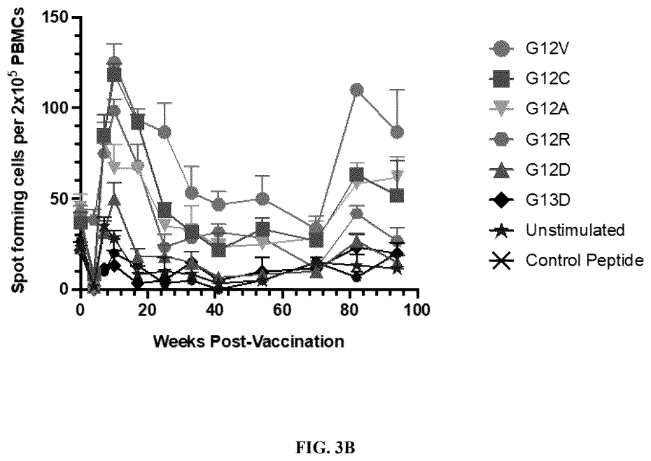

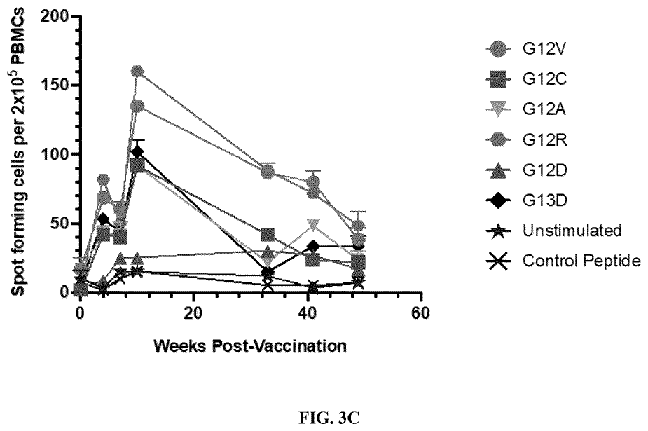

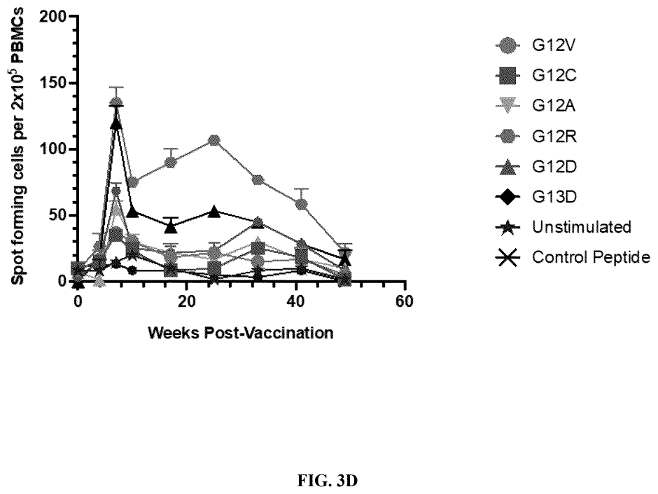

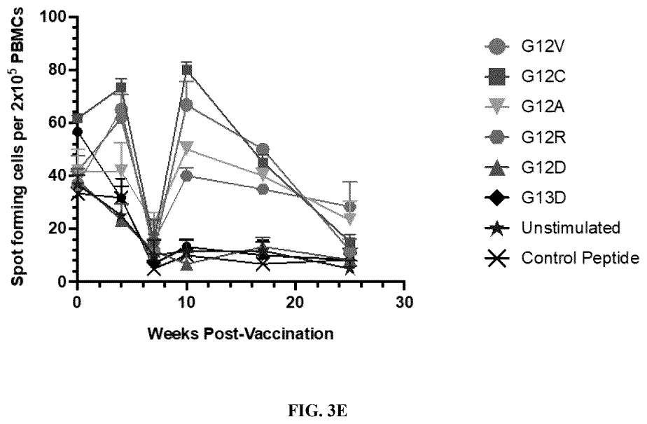

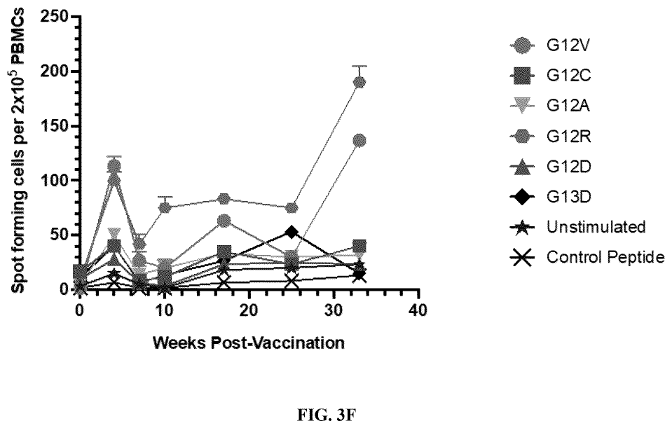

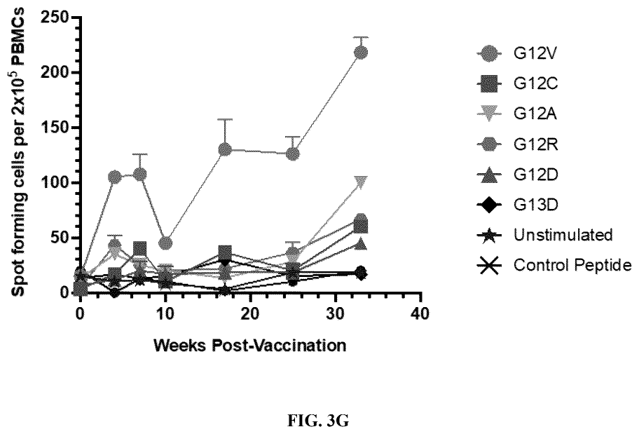

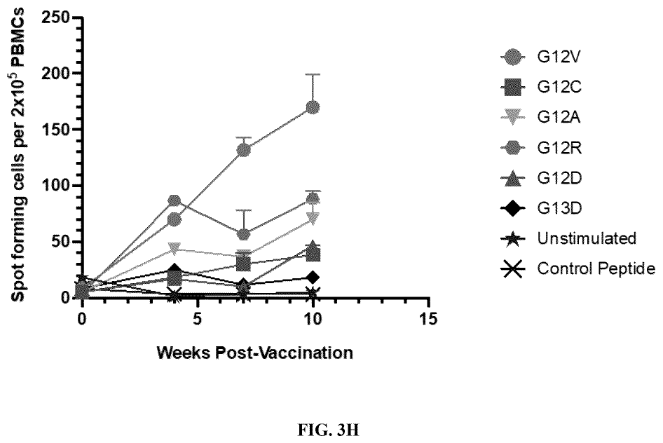

Immunogenicity of a mutant KRAS (mKRAS) vaccine in combination with nivolumab and ipilimumab were tested in twelve patients with resected pancreatic cancer who have completed peri-operative chemotherapy with or without radiation treatment. Longitudinal ELISpot data available from the available eleven PDAC patients demonstrates the induction of robust de novo mKRAS specific T cell responses against many of the peptides included in the vaccine including the ones expressed in each patient's respective tumor (FIGS. 2A-2K and FIGS. 3A-3H).

Enrolled patients received mKRAS vaccine plus ipilimumab (1 mg/kg, i.v., every 6 weeks for 2 doses) and nivolumab (3 mg/kg, i.v., every 3 weeks in priming phase followed by nivolumab (480 mg, i.v., flat dose in boost phase). Prime vaccines (0.3 mg for each synthetic long peptide, pooled) was given s.c. on days 1, 8, 15, and 22. During the booster phase, patients received vaccine on weeks 12, 20, 28, 36, and 44. Blood was drawn at timed intervals and peripheral PBMCs were subjected to IFNγ ELISpot. PBMCs were restimulated overnight with 2 ug/mL of individual KRAS G12V (SEQ ID NO: 3), G12A (SEQ ID NO: 5), G12R (SEQ ID NO: 7), G12C (SEQ ID NO: 2), G12D (SEQ ID NO: 4), or G13D (SEQ ID NO: 6) peptides. Unstimulated or control-peptide-stimulated PBMCs were used as negative controls. There was a notable induction of de novo, robust mutant-KRAS-specific T cell responses against most of the peptides included in the vaccine, including the ones expressed in each patient's tumor (FIGS. 2A-2K and FIGS. 3A-3H).

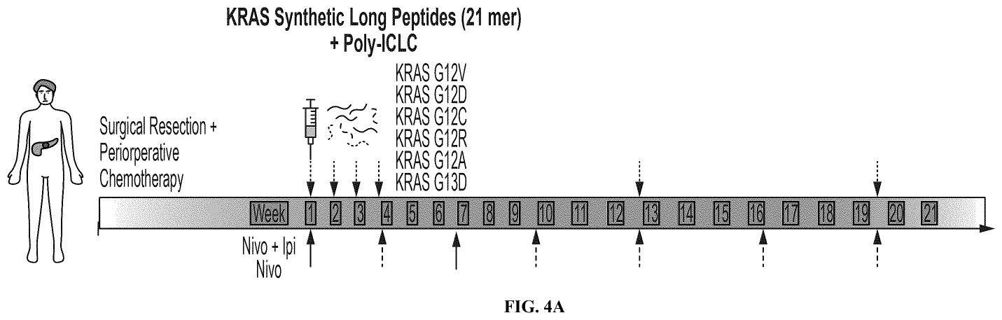

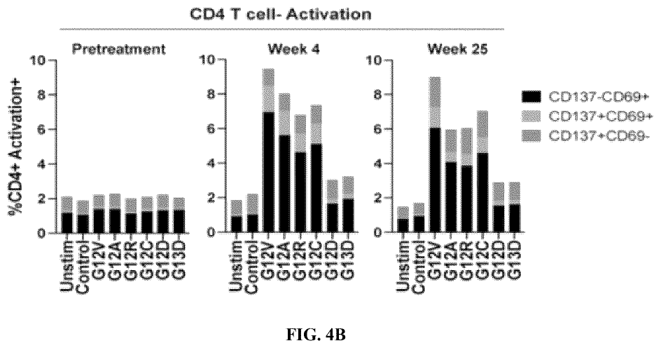

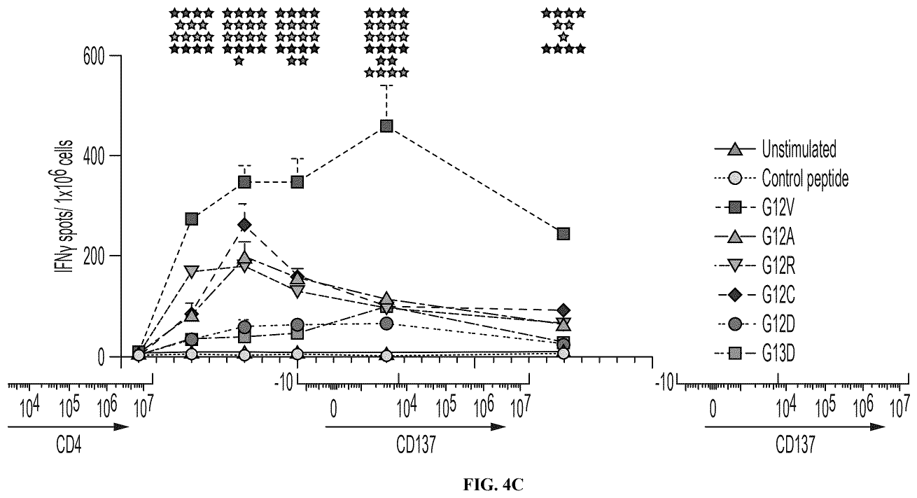

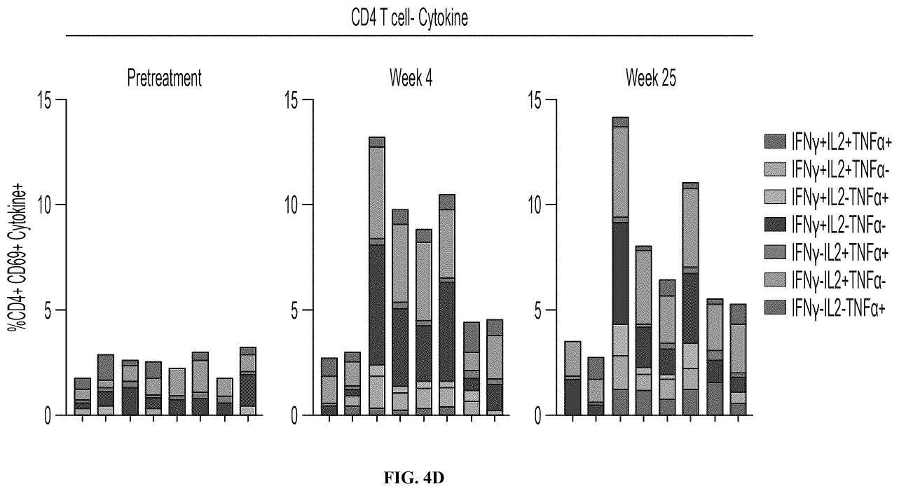

mKRAS peptide vaccine induces activated and polyfunctional mKRAS-Specific CD4 and CD8 T Cell Responses (FIGS. 4A-4D). PBMCs from a representative patient enrolled was subjected to flow cytometry to study T cell activation markers and cytokine expression. PBMCs were stimulated with 2 μg/mL of individual KRAS peptides (G12V, G12A, G12R, G12C, G12D, or G13D) for 48 hr. Flow cytometry was performed to assess for activation marker (CD69, and CD137) and cytokine (IFN [symbol] , IL-2, and TNFa ) expression in CD4 and CD8 T cells pre-and post-vaccination (FIG. 4D).

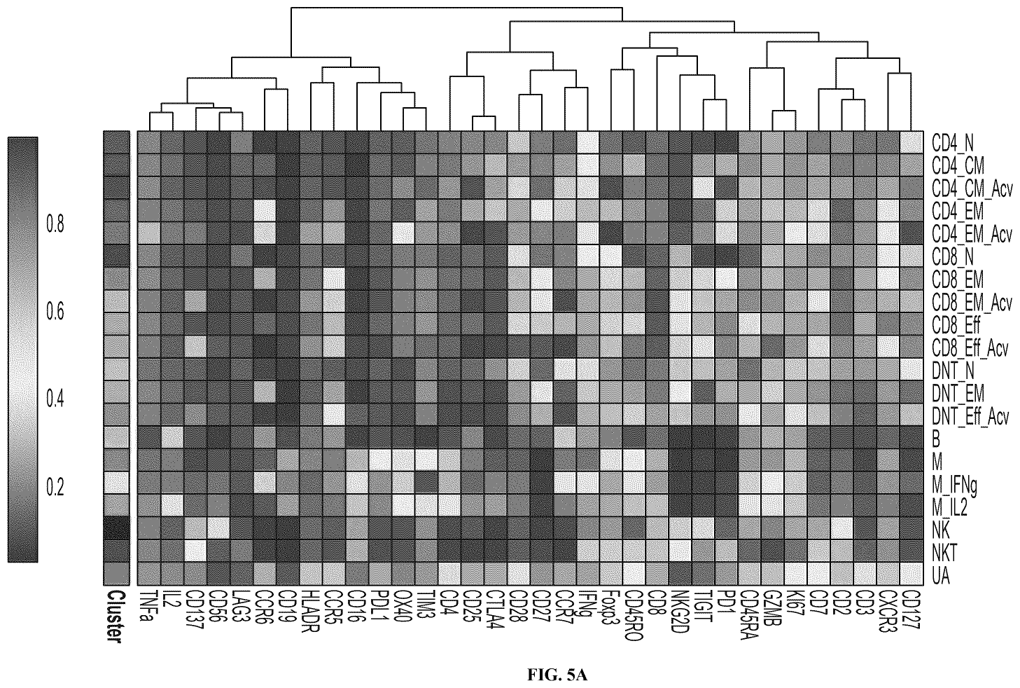

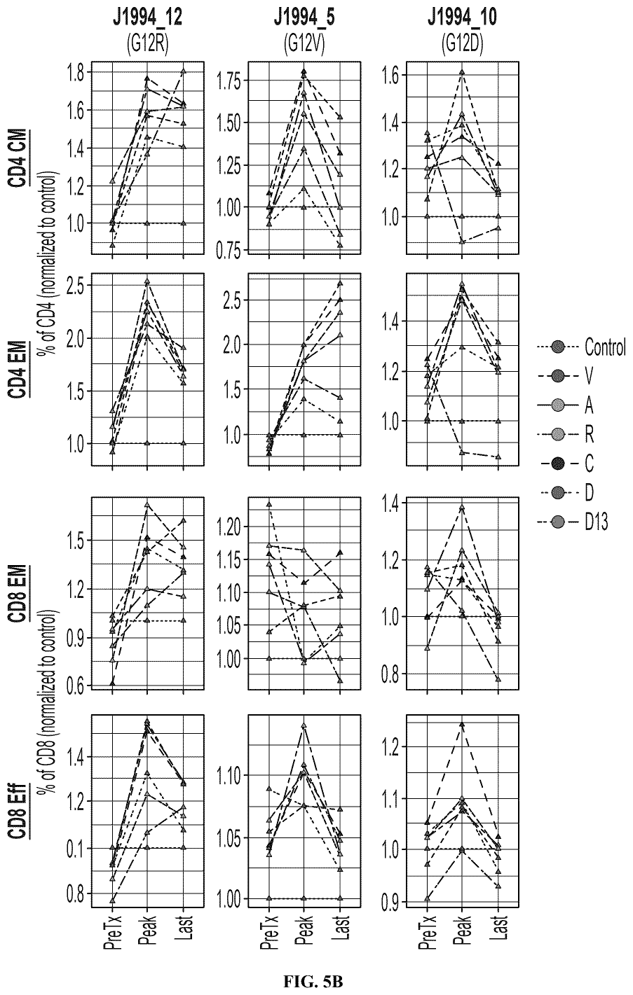

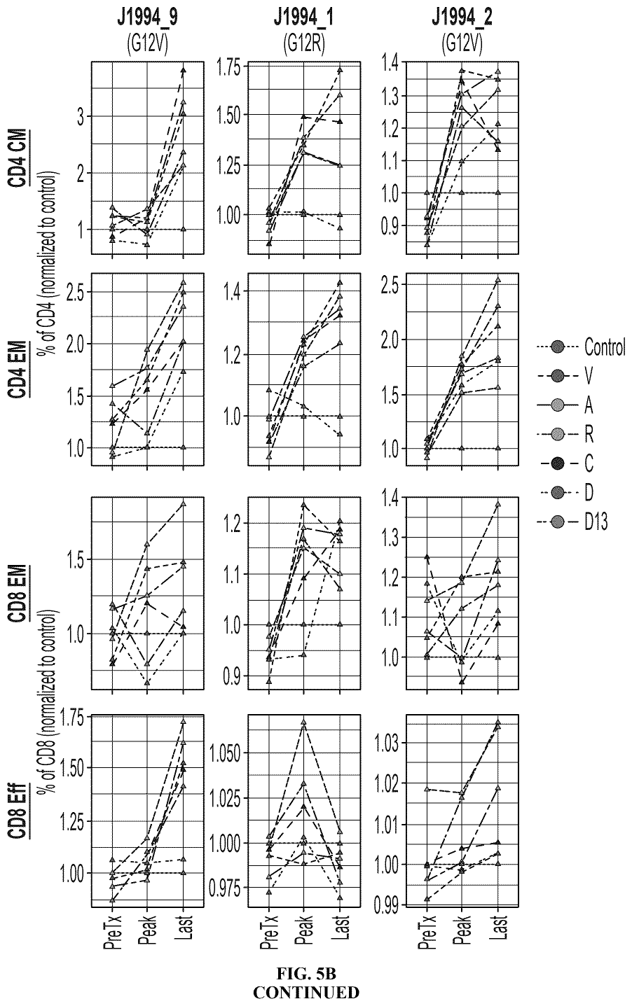

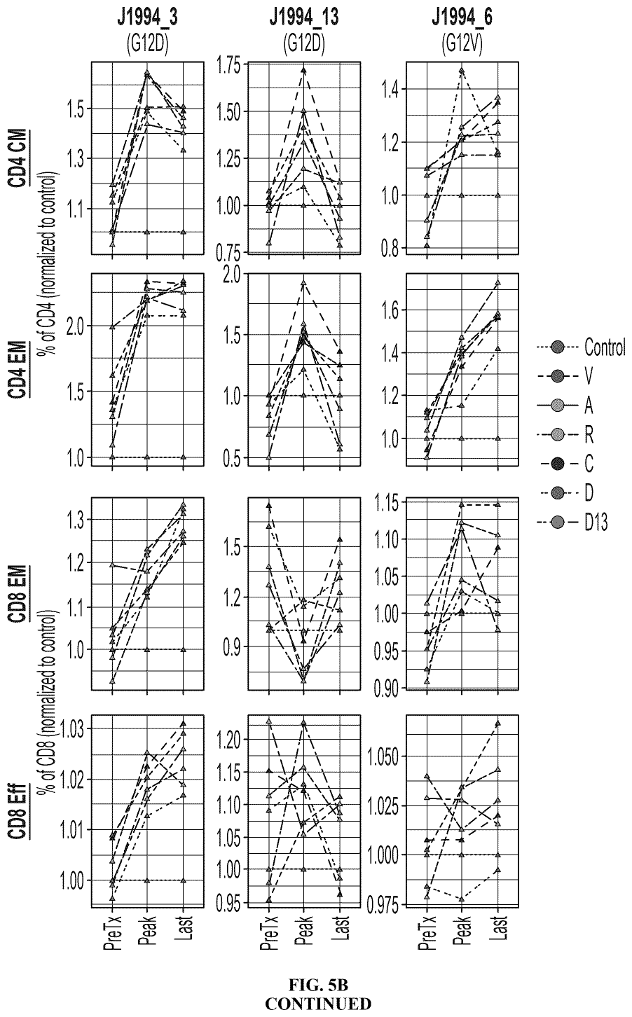

Vaccine-induced mKRAS-specific T cell responses also showed distinct CD4 and CD8 memory phenotypes. In FIG. 5A, heatmap of immune markers detected by CyTOF from PBMCs restimulated with 4 ug/mL control peptide, KRAS G12V, G12A, G12R, G12C, G12D, G13D SLP overnight, 37 ° C. In FIG. 5B, frequency of KRAS-responsive CD4 central memory (CM) and effector memory (EM) T cells or CD8 effector memory (EM) and effector (Eff) at pretreatment (PreTX), peak response (Peak), and last timepoint on trial (Last). Populations are normalized relative to control peptide. Each patient identifier J1994.XX and expressed mutation (G12X) is indicated.

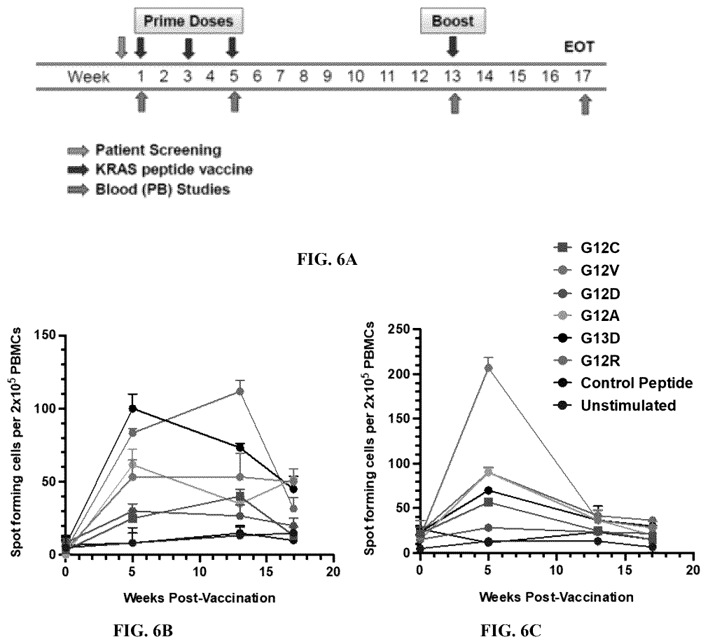

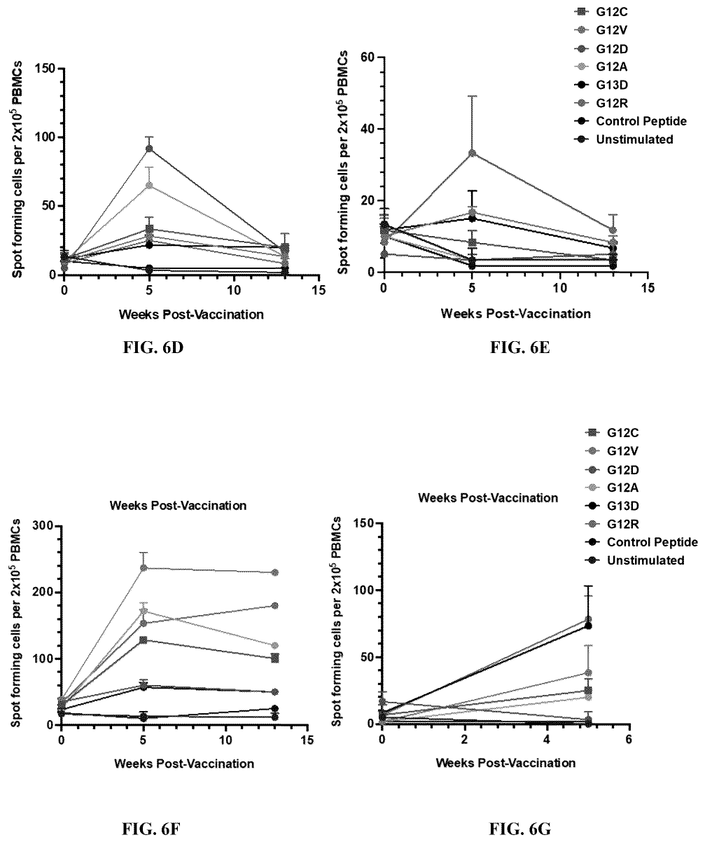

In addition, immunogenicity data testing mutant KRAS peptide vaccine in subjects who are at high risk of developing pancreatic cancer. This study tests the mutant KRAS peptide vaccine in subjects who are at high risk of developing pancreatic cancer due to family history and genetic predisposition. They are vaccinated per schema in FIG. 6A, and peripheral blood was collected per the indicated timepoints below. ELISPOT data were generated from the first five patients enrolled demonstrating T cell responses to the mKRAS antigens included in the vaccine (FIGS. 6B-6G).

FIGS. 6A-6G show trial design and preliminary immune results from prevention vaccine trial. Prime vaccines (0.3 mg for each synthetic long peptide, pooled) was given s.c. on week 1, 3, and 5 and booster vaccine was given on week 13 (FIG. 6A). Blood was drawn at timed intervals (as shown), and peripheral blood mononuclear cells (PBMCs) were subjected to IFNγ ELISpot. PBMCs were restimulated overnight with 2 ug/mL of individual KRAS G12V, G12A, G12R, G12C, G12D, or G13D peptides (FIGS. 6B-6G). Unstimulated or control-peptide-stimulated PBMCs were used as negative controls.

### Other Embodiments

From the foregoing description, it will be apparent that variations and modifications may be made to the disclosure described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference.

1.-70. (canceled)

71. A method of treating cancer in a subject in need thereof, the method comprising administering to the subject:
(a) a mutated KRAS peptide; and
(b) an anti-CTLA-4 chemotherapeutic agent or an anti-PD-1 chemotherapeutic agent.

72. The method of claim 71, wherein the mutated KRAS peptide comprises at least 10 amino acid residues.

73. The method of claim 71, wherein the mutated KRAS peptide comprises at least 15 amino acid residues.

74. The method of claim 71, wherein the mutated KRAS peptide comprises at least 20 amino acid residues.

75. The method of claim 71, wherein the mutated KRAS peptide comprises at least 21 amino acid residues.

76. The method of claim 71, wherein the mutated KRAS peptide comprises a G12C mutation, G12V mutation, a G12D mutation, a G12A mutation, a G12R mutation, a G13D mutation or combinations thereof.

77. The method of claim 71, wherein the mutated KRAS peptide comprises a sequence having at least 90% sequence identity to any one of SEQ ID NOs: 2-7.

78. The method of claim 77, wherein the mutated KRAS peptide comprises the sequence of any one of SEQ ID NOs: 2-7.

79. The method of claim 71, further comprising administering to the subject polyinosinic: polycytidylic acid (poly(I:C), poly-ICLC, derivatives thereof, or combinations thereof.

80. The method of claim 71, further comprising administering to the subject an adjuvant.

81. The method of claim 71, wherein the anti-CTLA-4 chemotherapeutic agent is an anti-CTLA-4 antibody.

82. The method of claim 81, wherein the anti-CTLA-4 antibody is selected from a group consisted of: Ipilimumab, Tremilimumab, Belatacept, and Abatacept.

83. The method of claim 71, wherein the anti-PD-1 chemotherapeutic agent is an anti-PD-1 antibody.

84. The method of claim 83, wherein the anti-PD-1 antibody is Nivolumab or Pembrolizumab.

85. The method of claim 71, wherein the anti-CTLA-4 chemotherapeutic agent comprises Ipilimumab and the anti-PD-1 chemotherapeutic agent comprises Nivolumab.

86. The method of claim 71, wherein the cancer is pancreatic cancer.

87. The method of claim 71, wherein the cancer is colon cancer or lung cancer.

88. An immunogenic composition comprising:
(a) a mutated KRAS peptide; and
(b) an anti-CTLA-4 chemotherapeutic agent or an anti-PD-1 chemotherapeutic agent.

89. A pharmaceutical composition comprising the immunogenic composition of claim 88 and a pharmaceutically acceptable carrier, excipient, or additive.

90. A method of inducing an immune response to a neoantigen in a subject in need thereof, comprising administering to the subject an effective amount of an immunogenic composition comprising:
(a) a mutated KRAS peptide; and
(b) an anti-CTLA-4 chemotherapeutic agent or an anti-PD-1 chemotherapeutic agent.
